(19) United States
(12) Patent Application Publication
(10) Pub. No.: US 2007/0248540 A1
(43) Pub. Date: Oct. 25, 2007

(54) Biochemical methods for measuring metabolic fitness of tissues or whole organisms

(75) Inventor: Marc K. Hellerstein, Kensington, CA (US)

Correspondence Address: MORRISON & FOERSTER LLP, 425 MARKET STREET, SAN FRANCISCO, CA, 94105-2482, US

(73) Assignee: The Regents of the University of California, Oakland, CA (US)

(21) Appl. No.: 11/796,438

(22) Filed: Apr. 26, 2007

Related U.S. Application Data
(63) Continuation-in-part of application No. 10/664,513, filed on Sep. 16, 2003, now abandoned.
(60) Provisional application No. 60/411,029, filed on Sep. 16, 2002.

(51) Int. Cl. A61K 51/00; A61K 49/00; C12Q 1/00

(52) U.S. Cl. 424/1.61; 424/1.11; 435/4

(57) ABSTRACT

The present invention relates to biochemical methods for assessing metabolic fitness and/or aerobic demands of a living system. Specifically, the rate of synthesis and turnover of the molecular components of mitochondrial mass are used to determine the aerobic capacity and/or aerobic demand of tissues or living organisms. The direct measurement of metabolic fitness and/or aerobic demand by this means can be used as an index of the efficacy of an exercise training program or other therapeutic intervention; as a medical risk factor for predicting the risk of cardiovascular disease, diabetes, death or other health outcome; or as an aid to pharmaceutical companies for drug discovery in the area of metabolic fitness, deconditioning, and oxidative biology.

### CROSS-REFERENCE TO RELATED APPLICATIONS

This application is a continuation-in-part of U.S. application Ser. No. 10/664,513, filed Sep. 16, 2003, which claims the benefit of U.S. Provisional Application No. 60/411,029 filed Sep. 16, 2002, both of which are hereby incorporated by reference in their entirety.

### FIELD OF THE INVENTION

The present invention is directed to the field of oxidative biology. In particular, methods for determining metabolic fitness by measuring the synthesis rates of mitochondrial DNA, RNA, proteins, or phospholipids are described.

### BACKGROUND OF THE INVENTION

The level of physical fitness (metabolic fitness, cardiorespiratory fitness) in humans has been shown to be a strong predictor for heart disease, diabetes, and overall mortality. Recent epidemiologic studies suggest that physical fitness instead of body fatness may be the most accurate risk factor in predicting all-cause mortality (Blair et al., Changes in Physical Fitness and All-Cause Mortality, JAMA 273(14):1093-1098 (1998) and Lee et al., Cardiorespiratory Fitness, Body Composition, and All-Cause and Cardiovascular Disease Mortality in Men, Am J Clin Nutr 69(3):373-380 (1999)). Support for this conclusion is evidenced by data demonstrating that some individuals who are overweight but fit metabolically exhibit a better health prognosis than individuals who are thin but unfit metabolically (see Lee et al., supra). Thus, being overweight may primarily serve as a marker for an underlying sedentary lifestyle and metabolically unfit state, rather than being the true risk-factor itself.

These findings have potentially profound clinical and public health implications. A physician's focus on the body fat of a patient may be misplaced if the key variable to monitor is metabolic fitness. Similarly, pharmaceutical companies looking for drugs that improve health might be better advised to work on agents that increase tissue oxidative (aerobic) capacity than on agents that reduce body fat content. However, currently available methods for assessing the metabolic fitness of whole organisms, e.g., exercise testing, are crude, non-biochemical, poorly reproducible, and difficult to perform.

For example, exercise testing requires an individual to exercise on equipment such as a treadmill or stationary bike, with continuous electrocardiographic and blood pressure monitoring. Typically, exercise is continued under a controlled program until the individual is unable to continue or until 85% of the individual's maximal heart rate is achieved (Hutter, A. M., Jr. (1991). “Ischemic Heart Disease: Angina Pectoris,” Section 1 in Scientific American Medicine. E. Rubenstein and D. D. Federman eds., Scientific American, Inc., p. 4). With such a protocol, it can be easily seen that numerous factors including mental illness, physical impairments due to such afflictions as respiratory or muscle disease, and inconsistent physical effort by the patient may affect test results. Moreover, there is some potential risk associated with this protocol (i.e., the exertion required). Furthermore, exercise testing is characterized by wide inter-observer variability (due to differences in supervisors' performance and difficulty in standardization) and use of bulky equipment that is not easily stored in a medical office.

Therefore, new methods that are more convenient for outpatient use and which objectively and reliably determine metabolic fitness are needed.

### SUMMARY OF THE INVENTION

The present disclosure relates to biochemical methods for assessing metabolic fitness and/or aerobic demands of a living system. Specifically, the rate of synthesis and turnover of the molecular components of mitochondria in aerobic tissues such as skeletal muscle or heart are used to determine the aerobic capacity and/or aerobic demand of the tissues or the living organisms. The direct measurement of metabolic fitness and/or aerobic demand by this means can be used as an index of the efficacy of an exercise training program or other therapeutic intervention; as a medical biomarker or risk factor for predicting the risk of cardiovascular disease, diabetes, death or other health outcomes; or as an aid to pharmaceutical companies for drug discovery in the area of metabolic fitness, deconditioning, and oxidative biology.

Described herein are methods for assessing the metabolic fitness or aerobic demand of a living system. In one aspect, the method includes the steps of: a) administering ²H₂O to a living system in a manner sufficient to achieve a ²H₂O enrichment level of 0.5% to 15% of the total body water in the living system; b) allowing sufficient time for the ²H label to be incorporated into a mitochondrial molecule in the living system; c) measuring the isotopic content, isotopic pattern, rate of change of isotopic content, or rate of change of the isotopic pattern, of the mitochondrial molecule; and c) calculating the rate of synthesis or degradation of the mitochondrial molecule to assess the metabolic fitness or the aerobic demand of the living system.

In one embodiment, the ²H₂O enrichment level achieved is 1% to 10% of the total water in the living system. In another embodiment, the ²H₂O is administered orally to the living system as two or more doses over time.

The mitochondrial molecule of the methods described herein may be any mitochondrial molecule. In one embodiment, the mitochondrial molecule may be a deoxyribonucleic acid (DNA) molecule. In yet another embodiment, the mitochondrial molecule is a ribonucleic acid (RNA) chosen from ribosomal RNA, transfer RNA, and messenger RNA. The mitochondrial molecule may also be a protein chosen from a subunit of cytochrome c oxidase, a subunit of F₀ ATPase, a subunit of F₁ ATPase, a subunit of cytochrome c reductase, and a subunit of NADH-CoQ reductase. In one embodiment, the mitochondrial molecule is a phospholipid chosen from cardiolipin, phosphatidylcholine, phosphatidylethanolamine, and a mixture thereof.

In an embodiment of the methods described, the living system is chosen from skeletal muscle tissue cardiac muscle tissue, and adipose tissue. In another embodiment, the living system is a mammal such as a rodent or a human. In yet another embodiment, the living system is a cell chosen from a platelet and a cultured cell in a high-throughput screening assay system

In an embodiment of the method for assessing the metabolic fitness or aerobic demand of a living system, the step of measuring isotopic content, pattern or rate of change of isotopic content, or pattern is performed by mass spectroscopy, NMR spectroscopy, or liquid scintillation counting.

Also described herein are methods for identifying a drug agent capable of altering metabolic fitness or aerobic demand of a living system.

In one embodiment, the method for identifying a drug agent capable of altering metabolic fitness or aerobic demand of a living system includes the steps of: a) assessing the metabolic fitness or aerobic demand of the living system using the methods described herein; b) administering the drug agent to the living system; and c) re-assessing the metabolic fitness or aerobic demand of the living system according to the methods described herein after the administration of the drug; and d) comparing the metabolic fitness or aerobic demand of the living system before and after administration of the drug, wherein a change in the metabolic fitness or aerobic demand identifies the drug agent as capable of altering the metabolic fitness or aerobic demand of the living system.

In another embodiment of the method of identifying a drug agent capable of altering metabolic fitness or aerobic demand of a first living system includes the steps of: a) assessing the metabolic fitness or aerobic demand, according to the methods described herein, of a first living system to which the drug agent has been administered; b) assessing the metabolic fitness or aerobic demand, according to the methods described herein, of a second living system to which the drug agent has not been administered; and c) comparing the metabolic fitness or aerobic demand of the first living system and of the second living system, wherein a change in the metabolic fitness or aerobic demand of the first and second living systems identifies the drug agent as capable of altering the metabolic fitness or aerobic demand of the first living system.

Also described herein are methods for assessing deconditioning of a living system. In one embodiment, the method includes the steps of: a) administering an isotopically labeled precursor molecule to the living system; b) allowing for a period of time sufficient for the label of the isotopically labeled precursor molecule to be incorporated into a mitochondrial molecule in the living system; c) measuring the isotopic content, isotopic pattern, rate of change of isotopic content, or rate of change of isotopic pattern of the isotopically labeled precursor molecule in the living system; d) calculating the rate of synthesis or degradation of the isotopically labeled precursor molecule to assess the initial metabolic fitness or aerobic demand of the living system; e) subjecting the living system to a deconditioning event, resulting in a deconditioned living system; f) administering the isotopically labeled precursor molecule to the deconditioned living system; g) allowing for a period of time sufficient for the label of the isotopically labeled precursor molecule to be incorporated into a mitochondrial molecule in the deconditioned living system; h) measuring the isotopic content, isotopic pattern, rate of change of isotopic content, or rate of change of isotopic pattern of the isotopically labeled precursor molecule in the deconditioned living system; and i) calculating the rate of degradation of the isotopically labeled precursor molecule to assess the deconditioning of the deconditioned living system.

In an aspect of the method for assessing deconditioning of a living system, the isotopically labeled precursor molecule is labeled with a stable isotope chosen from ²H-labeled glucose, ¹³C-labeled glucose, a ²H-labeled amino acid, a ¹⁵N-labeled amino acid, a ¹³C-labeled amino acid, ²H-labeled acetate, ¹³C-labeled acetate, a ²H-labeled ribonucleoside, a ¹³C-labeled ribonucleoside, a ¹⁵N-labeled ribonucleoside, a ²H-labeled deoxyribonucleoside, a ¹³C-labeled deoxyribonucleoside, a ¹⁵N-labeled deoxyribonucleoside, a ²H-labeled fatty acid, and a ¹³C-labeled fatty acid. In one embodiment, the isotopically labeled precursor molecule is ²H₂O that is administered orally as two or more doses over time.

In another aspect of the method for assessing deconditioning of a living system, the isotopically labeled precursor molecule is labeled with a radioactive isotope chosen from ³H-labeled glucose, ¹⁴C-labeled glucose, a ³H-labeled amino acids, a ¹⁴C-labeled amino acid, ³H-labeled acetate, ¹⁴C-labeled acetate, a ³H-labeled ribonucleoside, a ¹⁴C-labeled ribonucleoside, a ³H-labeled deoxyribonucleoside, a ¹⁴C-labeled deoxyribonucleoside, a ³H-labeled fatty acid, and a ¹⁴C-labeled fatty acid.

The mitochondrial molecule of the method for assessing deconditioning of a living system may be a deoxyribonucleic acid (DNA) molecule. In another embodiment, the mitochondrial molecule is a ribonucleic acid (RNA) molecule chosen from ribosomal RNA, transfer RNA, and messenger RNA. In yet another embodiment, the mitochondrial molecule is a protein chosen from a subunit of cytochrome c oxidase, a subunit of F₀ ATPase, a subunit of F₁ ATPase, a subunit of cytochrome c reductase, and a subunit of NADH-CoQ reductase. In another embodiment, the mitochondrial molecule is a phospholipid chosen from cardiolipin, phosphatidylcholine, phosphatidylethanolamine, and a mixture thereof.

The living system of the method for assessing deconditioning of a living system may be chosen from skeletal muscle tissue, cardiac muscle tissue, and adipose tissue. In another embodiment, the living system is a mammal such as a rodent or a human. In yet another embodiment, the living system is platelet or a cultured cell in a high-throughput screening assay system

In an embodiment of the method for assessing deconditioning of a living system, the step of measuring isotopic content, pattern or rate of change of isotopic content, or pattern is performed by mass spectroscopy, NMR spectroscopy, or liquid scintillation counting.

Described herein is an isolated, ²H labeled mitochondrial molecule with its ²H label derived from ²H labeled water (²H₂O). The isolated, ²H labeled mitochondrial molecule is produced by a) administering the ²H₂O to a living system, the ²H₂O is labeled with a detectable amount of ²H and is incorporated into the mitochondrial molecule of the living system, and b) isolating said ²H labeled mitochondrial molecule. Additionally, the isolated, ²H labeled mitochondrial molecule is a molecule chosen from DNA, RNA, a subunit of cytochrome c oxidase, a subunit of F₀ ATPase, a subunit of F₁ ATPase, a subunit of cytochrome c reductase, a subunit of NADH-CoQ reductase, cardiolipin, phosphatidylcholine, and phosphatidylethanolamine.

Also described is a kit for assessing the metabolic fitness or aerobic demand of a living system. The kit contains a) ²H₂O, and b) instructions for use of the kit and the kit is used to assess the metabolic fitness or aerobic demand of a living system.

### BRIEF DESCRIPTION OF THE DRAWINGS

FIG. 1A is an exemplary schematic of the protocol for isotopically labeled water (²H₂O) administration and sample collection for rats.

FIG. 1B illustrates the protocol for isotopically labeled water (2H₂0) administration and sample collection for human subjects.

FIG. 2A shows the increased incorporation of ²H from administered ²H₂O into mitochondrial DNA isolated from rats subjected to one week of exercise training as measured by gas chromatography/mass spectrometry.

FIG. 2B demonstrates the incorporation of ²H into mitochondrial DNA isolated from human muscle biopsies as measured by gas chromatography/mass spectrometry.

FIG. 3A shows the experimental protocol for the measurement of the rate of synthesis of mitochondrial DNA and mitochondrial phospholipids in human subjects, as measured from mitochondria isolated from muscle biopsies taken after the human subjects ingested ²H₂O.

FIG. 3B shows the effects of different exercise regimens on incorporation of ²H from administered ²H₂O into mitochondrial phospholipids.

FIG. 4A shows the increased incorporation of 2H from administered ²H₂O into cardiolipin (CL), phosphatidylcholine (PL), and phosphatidylethanolamine (PE) in mitochondria isolated from the hindlimb muscle of rats subjected to voluntary exercise.

FIG. 4B shows the increased incorporation of ²H from administered ²H₂O into cardiolipin (CL), phosphatidylcholine (PL), and phosphatidylethanolamine (PE) in mitochondria isolated from the heart muscle of rats subjected to chronic exercise.

FIG. 5 depicts the average cytochrome C oxidase subunit IV expression in hindlimb muscle from rats trained for 1,2, and 6 weeks (n=6 per time point) compared to controls (n=6 per time point). Data are ±S.D. * denotes statistical significance (p<0.05) versus control values.

FIG. 6 depicts Cytochrome C oxidase subunit IV expression from rats detrained for 4 weeks (n=6) compared to controls (n=6). Data are ±S.D. No significant differences between groups are present.

FIGS. 7a-b show the forced treadmill training exercise protocol in 8-week-old female Sprague Dawley rats.

FIG. 8 shows the effect of different durations of treadmill exercise on fractional synthesis of mtDNA from the red portion of the gastrocnemius muscle of 2-month-old female Sprague Dawley rats. Groups with different superscript letters are significantly different (p<0.05) by one-way ANOVA with Tukey's follow-up test. Values are means ±SE (n=5-6 per group).

FIGS. 9a-b show the effect of voluntary wheel running on body weights (a) and daily energy intake (b) of 3-month-old female Sprague Dawley rats exercised for 0 (Sed Cont)(n=10), 1 (n=4), 2 (n=10), 3(n=6), or 8 weeks (n=3). Values are means ±SE. Values are means ±SE. * P<0.05 vs. sedentary control at same time point. **P<0.001 vs. sedentary control at same time point.

FIG. 9c shows the average daily running distance of 3 month old female Sprague Dawley rats exercised 1 (n=4), 2 (n=10), 3(n=6), or 8 weeks (n=3). Values are means ±SE.

FIG. 9d shows the effect of different durations of voluntary exercise on fractional synthesis of mtDNA from the red portion of the gastrocnemius of female Sprague Dawley rats. Groups with different superscript letters are significantly different (p<0.05) by one-way ANOVA with Tukey's follow-up test. Values are means ±SE.

FIG. 9e shows effect of different durations of voluntary exercise on relative changes of cytochrome C oxidase activity from the red portion of the gastrocnemius of female Sprague Dawley rats. Values are means ±SE.

FIG. 10a shows the distance run by male and female Sprague Dawley rats consuming a 60% fat diet.

FIG. 10b shows the effect of voluntary exercise and high fat diet on fractional synthesis of mtDNA from the red and white portions of the gastrocnemius of female SD rats.

FIG. 10c shows the effect of voluntary exercise and 60% fat diet on cytochrome C oxidase activity from the red and white portions of the gastrocnemius of female SD rats.

FIG. 10d shows the effect of voluntary exercise and 60% fat diet on fractional synthesis of mtDNA from the red and white portions of the gastrocnemius of male SD rats.

FIG. 10e shows the effect of voluntary exercise and 60% fat diet on cytochrome C oxidase activity from the red and white portions of the gastrocnemius of male SD rats.

FIG. 11a shows the distance run by 10 week old female ZDF rats.

FIG. 11b shows the effect of voluntary activity on the fractional synthesis of mtDNA of the red and white portions of the gastrocnemius in 10 week old female ZDF rats.

FIG. 11c shows the effect of voluntary activity on cytochrome C oxidase activity of the red and white portions of the gastrocnemius in 10 week old female ZDF rats.

FIG. 12a shows the distance run by 10 week old female Fa/Fa rats.

FIG. 12b shows the effect of voluntary activity on the fractional synthesis of mtDNA of the red portion of the gastrocnemius in 10 week old female Fa/Fa rats.

FIG. 12c shows the effect of voluntary activity on the fractional synthesis of mtDNA of the white portion of the gastrocnemius in 10 week old female Fa/Fa rats.

FIG. 12d shows the effect of voluntary activity on cytochrome C oxidase activity of the red and white portions of the gastrocnemius in 10 week old female Fa/Fa rats.

FIG. 12e shows the correlation of distance run compared to the fractional synthesis of mtDNA from the red portion of the gastrocnemius of female Fa/Fa rats.

### DETAILED DESCRIPTION OF THE INVENTION

The present invention provides methods for the biochemical assessment of metabolic fitness (both increases in fitness and deconditioning) by measuring the rate of mitochondrial synthesis or degradation of mitochondrial molecules such as deoxyribonucleic acids (DNA), ribonucleic acids (RNA), proteins, or lipids in mitochondria of tissues. The rate of synthesis or degradation is based on the isotopic content and/or pattern or the rate of change of the isotopic content and/or pattern in mitochondrial molecules measured after administration of, or contact with, one or more isotopically labeled precursor molecules, including isotopically labeled water, where the isotope label is incorporated into mitochondrial molecules.

General Techniques

Practice of the present invention will generally utilize, unless otherwise indicated, conventional techniques of molecular biology, microbiology, cell biology, biochemistry, and immunology, which are within the skill of the art. Such techniques are fully explained in the literature, for example, in Cell Biology: A Laboratory Notebook (J. E. Cellis, ed., 1998); Current Protocols in Molecular Biology (F. M. Ausubel et al., eds, 1987); Short Protocols in Molecular Biology (Wiley and Sons, 1999); Mass Isotopomer Distribution Analysis: A Technique for Measuring Biosynthesis and Turnover of Polymers (Hellerstein et al., Am J Physiol 263 (Endocrinol Metab 26):E988-E1001 (1992)); and Mass Isotopomer Distribution Analysis at Eight Years: Theoretical, Analytic, and Experimental Considerations (Hellerstein et al., Am J Physiol 276 (Endocrinol Metab 39):E1146-1170 (1999)). Furthermore, procedures employing commercially available assay kits and reagents will typically be used according to manufacturer defined protocols unless otherwise noted.

Definitions

The terms “metabolic fitness”, “physical fitness”, and “cardiorespiratory fitness” herein are used interchangeably, and refer to the capacity for oxidative metabolism or aerobic activity of a living system.

By “living system” is meant herein any living entity including a cell, cell line, tissue, organ, and organism. The living system is preferably an organism. Examples of organisms include any animal, preferably a vertebrate, more preferably a mammal, most preferably a human. Examples of mammals include nonhuman primates, farm animals, pet animals, for example cats and dogs, and research animals, for example mice, rats, and humans. The human can be healthy or suffering from, or diagnosed with, a disease or disorder.

“Aerobic demand” refers to the oxidative needs imposed on a cell, tissue, or organism in vivo.

By “isotopes” it is meant herein atoms with the same number of protons and hence the same element but with different numbers of neutrons (e.g., ¹H vs. ²H or ³H). As is commonly known in the art, the symbol “D” is used interchangeably with the symbol ²H to refer to deuterium.

“Isotopically labeled precursor molecule” and “isotope labeled precursor molecule” are used interchangeably and refer to any isotope labeled precursor molecule from which the isotope label into a mitochondrial molecule in a living system. Examples of isotope labeled precursor molecules include, but are not limited to, ²H₂O, ³H₂O, ²H-glucose, ²H-labeled amino acids, ²H-labeled organic molecules, ¹³C-labeled organic molecules, ¹⁴C-labeled organic molecules, ¹³CO₂, ¹⁴CO₂, ¹⁵N-labeled organic molecules and ¹⁵NH₃.

“Isotopologues” refer to isotopic homologues or molecular species that have identical elemental and chemical compositions but differ in isotopic content (e.g., CH₃NH₂ vs. CH₃NHD in the example above). Isotopologues are defined by their isotopic composition; therefore each isotopologue has a unique exact mass but may not have a unique structure. An isotopologue is usually comprised of a family of isotopic isomers (isotopomers) which differ by the location of the isotopes on the molecule (e.g., CH₃NHD and CH₂DNH₂ are the same isotopologue but are different isotopomers).

“Exact mass” refers to mass calculated by summing the exact masses of all the isotopes in the formula of a molecule (e.g., 32.04847 for CH₃NHD).

“Nominal mass” refers to the integer mass obtained by rounding the exact mass of a molecule.

“Mass isotopomer” refers to family of isotopic isomers that is grouped on the basis of nominal mass rather than isotopic composition. A mass isotopomer may comprise molecules of different isotopic compositions, unlike an isotopologue (e.g., CH₃NHD, ¹³CH₃NH₂, CH₃¹⁵NH₂ are part of the same mass isotopomer but are different isotopologues). In operational terms, a mass isotopomer is a family of isotopologues that are not resolved by a mass spectrometer. For quadrupole mass spectrometers, this typically means that mass isotopomers are families of isotopologues that share a nominal mass. Thus, the isotopologues CH₃NH₂ and CH₃NHD differ in nominal mass and are distinguished as being different mass isotopomers, but the isotopologues CH₃NHD, CH₂DNH₂, ¹³CH₃NH₂, and CH₃¹⁵NH₂ are all of the same nomin mass and hence are the same mass isotopomers. Each mass isotopomer is therefore typically composed of more than one isotopologue and has more than one exact mass. The distinction between isotopologues and mass isotopomers is useful in practice because all individual isotopologues are not resolved using quadrupole mass spectrometers and may not be resolved even using mass spectrometers that produce higher mass resolution, so that calculations from mass spectrometric data must be performed on the abundances of mass isotopomers rather than isotopologues. The mass isotopomer lowest in mass is represented as Mo; for most organic molecules, this is the species containing all ¹²C, ¹H, ¹⁶O, ¹⁴N, etc. Other mass isotopomers are distinguished by their mass differences from M₀ (M1, M2, etc.). For a given mass isotopomer, the location or position of isotopes within the molecule is not specified and may vary (i.e., “positional isotopomers” are not distinguished).

“Mass isotopomer envelope” refers to the set of mass isotopomers comprising the family associated with each molecule or ion fragment monitored.

“Mass isotopomer pattern” refers to a histogram of the abundances of the mass isotopomers of a molecule. Traditionally, the pattern is presented as percent relative abundances where all of the abundances are normalized to that of the most abundant mass isotopomer; the most abundant isotopomer is said to be 100%. The preferred form for applications involving probability analysis, such as mass isotopomer distribution analysis (MIDA), however, is proportion or fractional abundance, where the fraction that each species contributes to the total abundance is used. The term “isotope pattern” may be used synonymously with the term “mass isotopomer pattern.”

“Monoisotopic mass” refers to the exact mass of the molecular species that contains all ¹H, ¹²C, ¹⁴N, ¹⁶O, ³²S, etc. For isotopologues composed of C, H, N, O, P, S, F, Cl, Br, and I, the isotopic composition of the isotopologue with the lowest mass is unique and unambiguous because the most abundant isotopes of these elements are also the lowest in mass. The monoisotopic mass is abbreviated as m0 and the masses of other mass isotopomers are identified by their mass differences from m0 (m1, m2, etc.).

“Isotopically perturbed” refers to the state of an element or molecule that results from the explicit incorporation of an element or molecule with a distribution of isotopes that differs from the distribution that is most commonly found in nature, whether a naturally less abundant isotope is present in excess (enriched) or in deficit (depleted).

“Isolating” refers to separating one component from one or more additional components in a mixture of components. For example, isolating a biochemical component refers to separating one biochemical components from a mixture of biochemical components. Small quantities of additional biochemical components may be present in the isolated biochemical component.

As used herein, the terms “precursor subunit,” “precursor molecule,” and “precursor” are used interchangeably to refer to the metabolic precursors used during polymeric synthesis of specific molecules. Examples of precursor subunits include acetyl CoA, ribonucleic acids, deoxyribonucleic acids, amino acids, glucose, and glycine.

“Labeled water” as used herein refers to water that contains isotopes. Examples of labeled water include ²H₂0, ³H₂0, and H₂¹⁸0. As used herein, the term “isotopically labeled water” is used interchangeably with “labeled water.”

“Isotopic content” refers to the content of isotopes in a molecule or population of molecules relative to the content in the molecule or population of molecules naturally (i.e., prior to administration or contacting of isotope labeled precursor subunits). The term “isotope enrichment” is used interchangeably with isotopic content herein.

“Isotopic pattern” refers to the internal relationships of isotopic labels within a molecule or population of molecules, e.g., the relative proportions of molecular species with different isotopic content, the relative proportions of molecules with isotopic labels in different chemical loci within the molecular structure, or other aspects of the internal pattern rather than absolute content of isotopes in the molecule.

“Molecular flux rate” refers to the rate of synthesis and/or breakdown of molecules within a cell, tissue, or organism. “Molecular flux rate” also refers to a molecule's input into or removal from a pool of molecules, and is therefore synonymous with the flow into and out of said pool of molecules.

“Oxidative metabolism” refers to the sum total of all energy-yielding biochemical transformations of fuels by a cell, tissue, organism, or other living system that ultimately require the involvement of molecular oxygen interacting with the oxidative phosphorylation apparatus (electron transport chain or respiratory enzyme system) in the cell, tissue, or organism.

“Drug agent,” “pharmaceutical agent,” and “pharmacological agent” are used interchangeably to refer to any chemical entities, known drug or therapy, approved drug or therapy, biological agent (e.g., gene sequences, poly or monoclonal antibodies, cytokines, and hormones). Drug agents include, but are not limited to, any chemical compound or composition disclosed in, for example, the 13th Edition of The Merck Index (a U.S. publication, Whitehouse Station, N.J., USA), incorporated herein by reference in its entirety.

“Mitochondrial molecule” refers to a molecule, such as a macromolecule, of a mitochondrion. Examples of mitochondrial molecules include, but are not limited to, DNA, RNA, proteins, lipids, and carbohydrates. The mitochondrial molecule may be synthesized or degraded within a mitochondrion, synthesized or degraded outside the mitochondrion, or imported into, or exported from, a mitochondrion. If a mitochondrial molecule is imported into a mitochondrion, then the mitochondrial molecule may or may not be further processed once within a mitochondrial space. In like manner, once a mitochondrial molecule is exported from a mitochondrion, that mitochondrial molecule may or may not be further processed.

“Deconditioning” refers to the decrease in metabolic fitness or aerobic capacity of a subject that occurs in response to inactivity, bed-rest, lack of exercise training, weightlessness (e.g., during space flight), or other behavior.

Mitochondrial Adaptation to Aerobic Demand

Mitochondria are the organelles of oxidative phosphorylation and are present in nearly all eukaryotic cells. The mitochondrial mass (i.e., the sum of mitochondrial components, including DNA, RNA, proteins, lipids, and other mitochondrial molecules) within a cell depends upon the cell type and a variety of physiologic factors. Although large differences in mitochondrial mass have been documented for different cell types, under resting conditions, the mitochondrial mass of each particular cell type is generally characteristic of the metabolic fitness of the cell. The mitochondrial mass generally reflects the capacity of a cell or tissue for oxidative metabolism or aerobic activity.

However, a change in aerobic demand, e.g., due to aerobic training placed upon a tissue such as skeletal or cardiac muscle has been identified as varying mitochondrial mass. In general, mitochondrial mass increases in response to aerobic exercise training programs, for example, and decreases in response to the deconditioning that occurs with inactivity such as bedrest. This adaptability of mitochondrial mass to the aerobic demand placed upon a tissue, thereby modulating the capacity of a tissue for oxidative metabolism (its aerobic capacity), is a fundamental characteristic of oxidative biology. Mitochondrial adaptability has profound implications for human health in the setting of the progressively more sedentary lifestyles associated with industrialization and urbanization, as is occurring internationally.

There are several unique features about the biochemistry of adaptive changes in tissue mitochondrial mass (Attardi et al., Biogenesis of Mitochondria, Ann Rev Cell Biol 4:289-333 (1988)). First, mitochondrial DNA is separate and distinct from the remainder of eukaryotic cellular DNA, which is present in the nucleus. Additionally, the mitochondrial genome is circular rather than arranged linearly within chromosomes in the nucleus, is small (16-20 kB in animals) compared to nuclear DNA, is almost completely lacking in introns, is synthesized using a different DNA polymerase (DNA polymerase y) than is present in the nucleus and is inherited maternally and independently of nuclear mitosis or meiosis. Moreover, mitochondrial DNA synthesis is linked to mitochondrial RNA synthesis: The former (DNA replication) depends upon priming by DNA-based RNA-transcription (Clayton D., Replication and Transcription of Vertebrate Mitochondrial DNA, Ann Rev Cell Biol 7:453-478 (1991)). This dependence of replication on transcription results in coordinate induction of increased mitochondrial DNA synthesis when the cell is signaling the need for more mitochondrial RNA synthesis. Finally, mitochondrial proteins and lipids are almost entirely derived from extra-mitochondrial synthesis, unlike mitochondrial DNA. Over 90% of mitochondrial proteins are synthesized from cytosolic messenger RNA templates which are in turn derived from nuclear DNA coding sequences. Proteins synthesized in the cytosol are then imported into mitochondria (see Lee et al. and Attardi et al., supra). Only a small number of (essential) enzymes involved in mitochondrial oxidative metabolism are coded by mitochondrial DNA. Most of the mitochondrial RNA transcripts derived from mitochondrial DNA are used for the protein synthetic apparatus (e.g., for ribosomal or transfer RNA), rather representing messenger RNA templates for protein synthesis.

It should also be noted that the model that discrete mitochondria exist and that there is a countable “mitochondrial number” is increasingly believed to be an oversimplification and incorrect (Robin et al., Mitochondrial DNA Molecules and Virtual Number of Mitochondria Per Cell in Mammalian Cells, J Cell Physiol 136:507-513 (1988)). Mitochondria in a cell are connected three-dimensionally through a reticulum that probably allows the flow of materials among the components. Because mitochondrial DNA exists as small circular genomes that are present at many copies per apparent mitochondrial “unit,” even the DNA content of the mitochondrial reticulum is probably exchangeable between and among components.

The currently available techniques for measuring mitochondrial mass or activity are all limited in one fundamental respect; namely, they are static in nature rather than reflecting dynamic processes. Typically, these techniques measure levels of such factors as mitochondrial oxidative enzymes (e.g., citrate synthase) or mitochondrial DNA or RNA content, which only reveals the concentration present at that moment in time. However, adaptations in mitochondrial mass in response to aerobic demands involve kinetic changes (i.e., changes in molecular flux rates, including the rates of synthesis or catabolism of mitochondrial components). There had been until recently, however, no way to assess the rates of synthesis or breakdown of mitochondrial components, and therefore, no way to assess the underlying dynamics of mitochondrial mass or the trajectory (the direction of change) of mitochondrial mass or mitochondrial dynamics in response to tissue oxidative demand. In one aspect, mitochondrial mass changes in response to the synthesis and/or degradation of mitochondrial molecules.

Methods For Assessing Metabolic Fitness

The present invention provides methods for assessing metabolic fitness (both increases in fitness and decreases in fitness) by measuring the rate of synthesis or degradation of various mitochondrial molecules. Examples of mitochondrial molecules include, but are not limited to DNA, RNA, lipids, carbohydrates, and proteins. RNA includes ribosomal RNA, transfer RNA, and messenger RNA. Lipids include phospholipids. Proteins include subunits of the various macromolecular complexes comprising the electron transport chain and involved in oxidative phosphorylation (aerobic respiration). These subunits include subunits of cytochrome c oxidase, subunits of F₀ ATPase, subunits of F₁ ATPase, subunits of cytochrome c reductase, and subunits of NADH-CoQ reductase.

In one aspect, a method is disclosed for assessing metabolic fitness or aerobic demand of a living system by administering an isotopically labeled precursor molecule to the living system time sufficient for the label of the isotopically labeled precursor molecule to be incorporated into a mitochondrial molecule; obtaining one or more mitochondrial molecules from the living system; measuring the isotopic content, isotopic pattern, rate of change of isotopic content, or rate of change of isotopic pattern of the mitochondrial molecule; and calculating the rate of synthesis or degradation of the mitochondrial molecule to assess metabolic fitness or aerobic demand of the living system.

In particular, the present invention provides methods for assessing deconditioning by measuring the rate of synthesis or degradation of various mitochondrial molecules. Exercise training generally improves the metabolic fitness of a subject. Subsequent inactivity for variable periods of time, typically at least approximately 2 weeks typically results in a decrease in metabolic fitness known as detraining or deconditioning.

Examples of the mitochondrial molecules measured to assess deconditioning include, but are not limited to DNA, RNA, lipids, carbohydrates, and proteins. RNA includes ribosomal RNA, transfer RNA, and messenger RNA. Lipids include phospholipids. Proteins include subunits of the various macromolecular complexes comprising the electron transport chain and involved in oxidative phosphorylation (aerobic respiration). These subunits include subunits of cytochrome c oxidase, subunits of F₀ ATPase, subunits of F₁ ATPase, subunits of cytochrome c reductase, and subunits of NADH-CoQ reductase.

In one aspect, a method is disclosed for assessing deconditioning of a living system by administering an isotopically labeled precursor molecule to the living system time sufficient for the label of the isotopically labeled precursor molecule to be incorporated into a mitochondrial molecule; obtaining one or more mitochondrial molecules from the living system; measuring the isotopic content, isotopic pattern, rate of change of isotopic content, or rate of change of isotopic pattern of the mitochondrial molecule; and calculating the rate of synthesis or degradation of the mitochondrial molecule to assess metabolic fitness or aerobic demand of the living system.

A. Administering to a Living System an Isotope-Labeled Precursor Molecule

1. Labeled Precursor Molecules

a. Isotope Labels

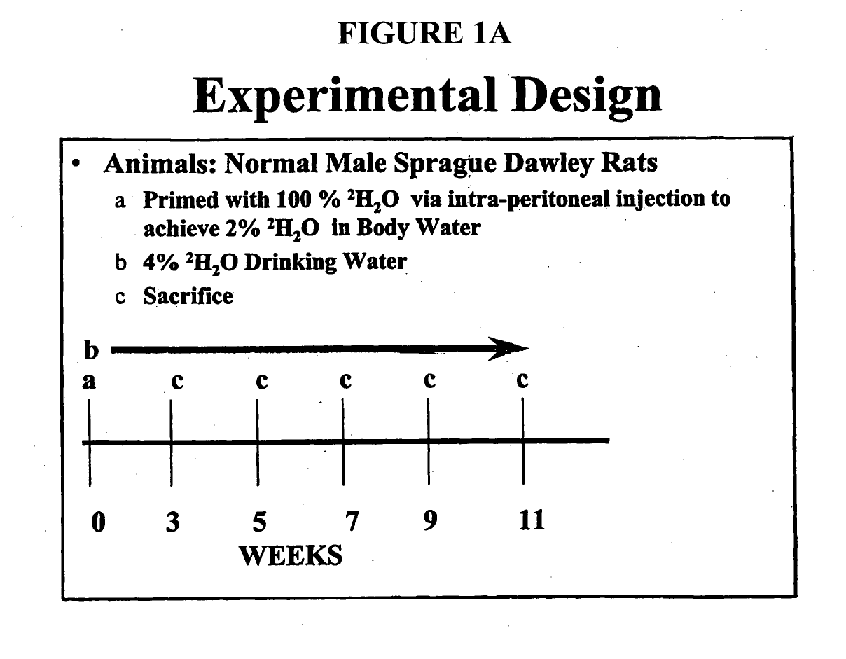

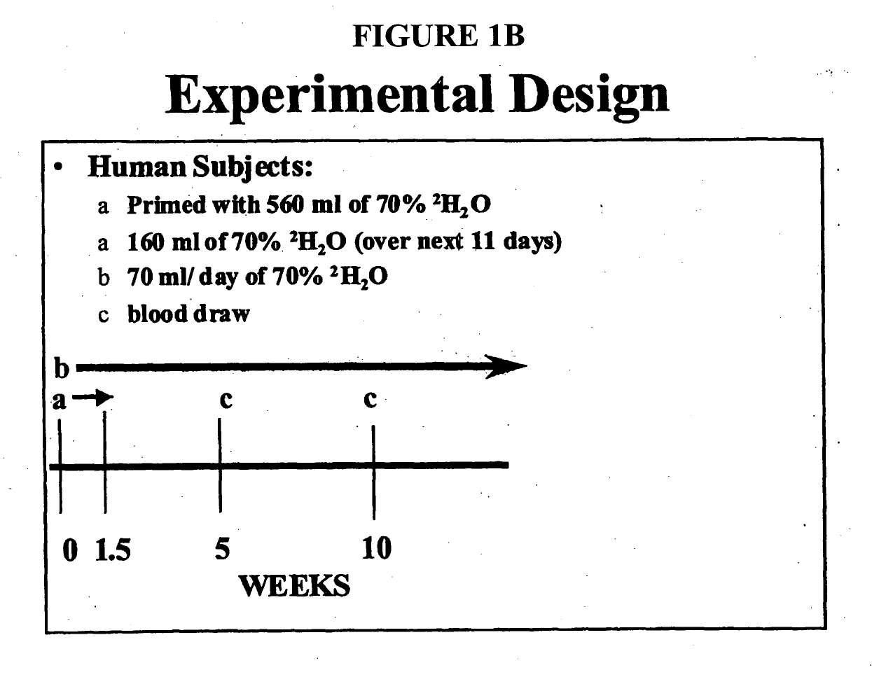

As illustrated in FIG. 1, the first step in measuring biosynthesis, breakdown, and/or turnover rates involve administering an isotope-labeled precursor molecule to a living system. The isotope labeled precursor molecule may be a stable isotope or radioisotope. Isotope labels that can be used include, but are not limited to, ²H, ¹³C, ¹⁵N, ¹⁸O, ³H, ¹⁴C, ³⁵S, ³²P, ¹²⁵I, ¹³¹I, or other isotopes of elements present in organic systems.

In one embodiment, the isotope label is ²H.

b. Precursor Molecules

The precursor molecule may be any molecule that is metabolized in the body to form a mitochondrial molecule. Isotope labels may be used to modify all precursor molecules disclosed herein to form isotope-labeled precursor molecules.

The entire precursor molecule may be incorporated into one or more mitochondrial molecules (e.g., mitochondrial molecules). Alternatively, a portion of the precursor molecule may be incorporated into one or more mitochondrial molecules.

Precursor molecules may include, but are not limited to, CO₂, NH₃, glucose, lactate, H₂O, acetate, fatty acids.

i. Water as a Precursor Molecule

Water is a precursor of proteins, polynucleotides, lipids, carbohydrates, modifications or combinations thereof, and other mitochondrial molecules. As such, labeled water may serve as a precursor in the methods taught herein.

Labeled water may be readily obtained commercially. For example, ²H₂O may be purchased from Cambridge Isotope Labs (Andover, Mass.), and ³H₂O may be purchased, e.g., from New England Nuclear, Inc. In general, ²H₂O is non-radioactive and thus, presents fewer toxicity concerns than radioactive ³H₂O. ²H₂O may be administered, for example, as a percent of total body water, e.g., 1% of total body water consumed (e.g., for 3 liters water consumed per day, 30 microliters ²H₂O is consumed). If ³H₂O is utilized, then a non-toxic amount, which is readily determined by those of skill in the art, is administered.

Relatively high body water enrichments of ²H₂O (e.g., 1-10% of the total body water is labeled) may be achieved using the techniques of the invention. Water enrichments of ²H₂O of 0.5%-15% may also be achieved using the techniques described herein. This water enrichment is relatively constant and stable as these levels are maintained for weeks or months in humans and in experimental animals without any evidence of toxicity. This finding in a large number of human subjects (>100 people) is contrary to previous concerns about vestibular toxicities at high doses of ²H₂O. Applicant has discovered that as long as rapid changes in body water enrichment are minimized (e.g., by initial administration in small, divided doses), high body water enrichments of ²H₂O can be maintained with a minimum toxicities. For example, the low expense of commercially available ²H₂O allows long-term maintenance of enrichments in the 1-5% range at relatively low expense (e.g., calculations reveal a lower cost for 2 months labeling at 2% ²H₂O enrichment, and thus 7-8% enrichment in the alanine precursor pool, than for 12 hours labeling of ²H-leucine at 10% free leucine enrichment, and thus 7-8% enrichment in leucine precursor pool for that period).

Relatively high and relatively constant body water enrichments for administration of H₂¹⁸O may also be accomplished, since the ¹⁸O isotope is not toxic, and does not present a significant health risk as a result.

Labeled water may be used as a near-universal precursor for most classes of mitochondrial molecules.

ii. Protein, Oligo/Polynucleotide, Lipid, and Carbohydrate Precursors

In another embodiment, precursor molecules are precursors of proteins, polynucleotides, lipids, and carbohydrates.

(a) Precursors of Proteins

The precursor molecule may be any protein precursor molecule known in the art. These precursor molecules may be CO₂, NH₃, glucose, lactate, H₂O, acetate, and fatty acids.

Precursor molecules of proteins may also include one or more amino acids. The precursor may be any amino acid. The precursor molecule may be a singly or multiply deuterated amino acid. The precursor molecule is one or more of ¹³C-lysine, ¹⁵N-histidine, ¹³C-serine, ¹³C-glycine, ²H-leucine, ¹⁵N-glycine, ¹³C-leucine, ²H₅-histidine, and any deuterated amino acid. Labeled amino acids may be administered, for example, undiluted with non-deuterated amino acids. All isotope labeled precursors may be purchased commercially, for example, from Cambridge Isotope Labs (Andover, Mass.).

The precursor molecule may also include any precursor for post-translational or pre-translationally modified amino acids. These precursors include but are not limited to precursors of methylation such as glycine, serine or H₂O; precursors of hydroxylation, such as H₂O or O₂; precursors of phosphorylation, such as phosphate, H₂O or O₂; precursors of prenylation, such as fatty acids, acetate, H₂O, ethanol, ketone bodies, glucose, or fructose; precursors of carboxylation, such as CO₂, O₂, H₂O, or glucose; precursors of acetylation, such as acetate, ethanol, glucose, fructose, lactate, alanine, H₂O, CO₂, or O₂; and other post-translational modifications known in the art.

The degree of labeling present in free amino acids may be determined experimentally, or may be assumed based on the number of labeling sites in an amino acid. For example, when using hydrogen isotopes as a label, the labeling present in C—H bonds of free amino acid or, more specifically, in tRNA-amino acids, during exposure to ²H₂O in body water may be identified. The total number of C—H bonds in each non essential amino acid is known—e.g., 4 in alanine, 2 in glycine, etc.

The precursor molecule for proteins may be water. The hydrogen atoms on C—H bonds are the hydrogen atoms on amino acids that are useful for measuring protein synthesis from ²H₂O since the O—H and N—H bonds of peptides and proteins are labile in aqueous solution. As such, the exchange of ²H-label from ²H₂O into O—H or N—H bonds occurs without the synthesis of proteins from free amino acids as described above. C—H bonds undergo incorporation from H₂O into free amino acids during specific enzyme-catalyzed intermediary metabolic reactions. The presence of ²H-label in C—H bonds of protein-bound amino acids after ²H₂O administration therefore means that the protein was assembled from amino acids that were in the free form during the period of ²H₂O exposure—i.e., that the protein is newly synthesized. Analytically, the amino acid derivative used must contain all the C—H bonds but must remove all potentially contaminating N—H and O—H bonds.

Hydrogen atoms from body water may be incorporated into free amino acids. ²H or ³H from labeled water can enter into free amino acids in the cell through the reactions of intermediary metabolism, but ²H or ³H cannot enter into amino acids that are present in peptide bonds or that are bound to transfer RNA. Free essential amino acids may incorporate a single hydrogen atom from body water into the α-carbon C—H bond, through rapidly reversible transamination reactions. Free non-essential amino acids contain a larger number of metabolically exchangeable C—H bonds, of course, and are therefore expected to exhibit higher isotopic enrichment values per molecule from ²H₂O in newly synthesized proteins

One of skill in the art will recognize that labeled hydrogen atoms from body water may be incorporated into other amino acids via other biochemical pathways. For example, it is known in the art that hydrogen atoms from water may be incorporated into glutamate via synthesis of the precursor α-ketoglutrate in the citric acid cycle. Glutamate, in turn, is known to be the biochemical precursor for glutamine, proline, and arginine. By way of another example, hydrogen atoms from body water may be incorporated into post-translationally modified amino acids, such as the methyl group in 3-methyl-histine, the hydroxyl group in hydroxyproline or hydroxylysine, and others. Other amino acids synthesis pathways are known to those of skill in the art.

Oxygen atoms (H₂¹⁸O) may also be incorporated into amino acids through enzyme-catalyzed reactions. For example, oxygen exchange into the carboxylic acid moiety of amino acids may occur during enzyme catalyzed reactions. Incorporation of labeled oxygen into amino acids is known to one of skill in the art. Oxygen atoms may also be incorporated into amino acids from ¹⁸O₂ through enzyme catalyzed reactions (including hydroxyproline, hydroxylysine or other post-translationally modified amino acids).

Hydrogen and oxygen labels from labeled water may also be incorporated into amino acids through post-translational modifications. In one embodiment, the post-translational modification may already include labeled hydrogen or oxygen through biosynthetic pathways prior to post-translational modification. In another embodiment, the post-translational modification may incorporate labeled hydrogen, oxygen, carbon, or nitrogen from metabolic derivatives involved in the free exchange labeled hydrogens from body water, either before or after post-translational modification step (e.g. methylation, hydroxylation, phosphoryllation, prenylation, sulfation, carboxylation, acetylation or other known post-translational modifications).

(b) Precursors of Oligo/Polynucleotides

The precursor molecule may include components of oligo or polynucleotides (oligonucleotide and polynucleotide used interchangeably in this context). Polynucleotides include purine and pyrimidine bases and a ribose-phosphate backbone. The precursor molecule may be any polynucleotide precursor molecule known in the art.

The precursor molecules of polynucleotides may be CO₂, NH₃, urea, O₂, glucose, lactate, H₂O, acetate, ketone bodies and fatty acids, glycine, succinate or other amino acids, and phosphate.

Precursor molecules of polynucleotides may also include one or more nucleoside residues. The precursor molecules may also be one or more components of nucleoside residues. Glycine, aspartate, glutamine, and tetryhydrofolate, for example, may be used as precursor molecules of purine rings. Carbamyl phosphate and aspartate, for example, may be used as precursor molecules of pyrimidine rings. Adenine, adenosine, guanine, guanosine, cytidine, cytosine, thymine, or thymidine may be given as precursor molecules for deoxyribonucleosides. All isotope labeled precursors may be purchased commercially, for example, from Cambridge Isotope Labs (Andover, Mass.).

The precursor molecule of polynucleotides may be water. The hydrogen atoms on C—H bonds of polynucleotides, polynucleosides, and nucleotide or nucleoside precursors may be used to measure polynucleotide synthesis from ²H₂O. C—H bonds undergo exchange from H₂O into polynucleotide precursors. The presence of ²H-label in C—H bonds of polynucleotides, nucleosides, and nucleotide or nucleoside precursors, after ²H₂O administration therefore means that the polynucleotide was synthesized during this period. The degree of labeling present may be determined experimentally, or assumed based on the number of labeling sites in a polynucleotide or nucleoside.

Hydrogen atoms from body water may be incorporated into free nucleosides or polynucleotides. ²H or ³H from labeled water can enter these molecules through the reactions of intermediary metabolism.

One of skill in the art will recognize that labeled hydrogen atoms from body water may be incorporated into other polynucleotides, nucleotides, or nucleosides via various biochemical pathways, for example, glycine, aspartate, glutamine, and tetryhydrofolate, which are known precursor molecules of purine rings. Carbamyl phosphate and aspartate, for example, are known precursor molecules of pyrimidine rings. Ribose and ribose phosphate, and their synthesis pathways, are known precursors of polynucleotide synthesis.

Oxygen atoms (H₂¹⁸O) may also be incorporated into polynucleotides, nucleotides, or nucleosides through enzyme-catalyzed biochemical reactions, including those listed above. Oxygen atoms from ¹⁸O₂ may also be incorporated into nucleotides by oxidative reactions, including non-enzymatic oxidation reactions (including oxidative damage, such as formation of 8-oxo-guanine and other oxidized bases or nucleotides).

Isotope-labeled precursors may also be incorporated into polynucleotides, nucleotides, or nucleosides in post-replication modifications. Post-replication modifications include modifications that occur after synthesis of DNA molecules. The metabolic derivatives may be methylated bases, including, but not limited to, methylated cytosine. The metabolic derivatives may also be oxidatively modified bases, including, but not limited to, 8-oxo-guanosine. Those of skill in the art will readily appreciate that the label may be incorporated during synthesis of the modification.

(c) Precursors of Lipids

Labeled precursors of lipids may include any precursor in lipid biosynthesis. The precursor molecules of lipids may be CO₂, NH₃, glucose, lactate, H₂O, acetate, and fatty acids. The precursor may also include labeled water, preferably ²H₂O (deuterated water), which is a precursor for fatty acids, glycerol moiety of acyl-glycerols, cholesterol and its derivatives; ¹³C or ²H-labeled fatty acids, which are precursors for triglycerides, phospholipids, cholesterol ester, coamides and other lipids; ¹³C— or ²H-acetate, which is a precursor for fatty acids and cholesterol; ¹⁸O₂, which is a precursor for fatty acids, cholesterol, acyl-glycerides, and certain oxidatively modified fatty acids (such as peroxides) by either enzymatically catalyzed reactions or by non-enzymatic oxidative damage (e.g. to fatty acids); ¹³C— or ²H-glycerol, which is a precursor for acyl-glycerides; ¹³C— or ²H-labeled acetate, ethanol, ketone bodies or fatty acids, which are precursors for endogenously synthesized fatty acids, cholesterol and acylglycerides; and ²H or ¹³C-labeled cholesterol or its derivatives (including bile acids and steroid hormones). All isotope labeled precursors may be purchased commercially, for example, from Cambridge Isotope Labs (Andover, Mass.).

Complex lipids, such as glycolipids and cerebrosides, can also be labeled from precursors, including ²H₂O, which is a precursor for the sugar-moiety of cerebrosides (including, but not limited to, N-acetylgalactosamine, N-acetylglucosamine-sulfate, glucuronic acid, and glucuronic acid-sulfate), the fatty acyl-moiety of cerebrosides and the sphingosine moiety of cerebrosides; ²H— or ¹³C-labeled fatty acids, which are precursors for the fatty acyl moiety of cerebrosides, glycolipids and other derivatives.

The precursor molecule may be or include components of lipids.

(d) Precursors of Glycosaminoglycans and Proteoglycans

Glycosaminoglycans and proteoglycans are a complex class of biomolecules that play important roles in the extracellular space (e.g. cartilage, ground substance, and synovial joint fluid). Molecules in these classes include, for example, the large polymers built from glycosaminoglycans disaccharides, such as hyaluronan, which is a polymer composed of up to 50,000 repeating units of hyaluronic acid (HA) disaccharide, a dimer that contains N-acetyl-glucosamine linked to glucuronic acid; chondroitin-sulfate (CS) polymers, which are built from repeating units of CS disaccharide, a dimer that contains N-acetyl-galactosamine-sulfate linked to glucuronic acid, heparan-sulfate polymers, which are built from repeating units of heparan-sulfate, a dimer of N-acetyl (or N-sulfo)-glucosamine-sulfate linked to glucuronic acid; and keratan-sulfate polymers, which are built from repeating units of keratan-sulfate disaccharide, a dimer that contains N-acetylglucosamine-sulfate liked to galactose. Proteoglycans contain additional proteins that are bound to a central hyaluronan in polymer and other glycosaminoglycans, such as CS, that branch off of the central hyaluronan chain.

Labeled precursors of glycosaminoglycans and proteoglycans include, but are not limited to, ²H₂O (incorporated into the sugar moieties, including N-acetylglucosamine, N-acetylgalactosamine, glucuronic acid, the various sulfates of N-acetylglucosamine and N-acetylgalactosamine, galactose, iduronic acid, and others), ¹³C— or ²H-glucose (incorporated into sugar moieties), ²H— or ¹³C-fructose (incorporated into the sugar moieties), ²H— or ¹³C-galactose (incorporated into said sugar moieties), ¹⁵N-glycine, other ¹⁵N-labeled amino acids, or ¹⁵N-urea (incorporated into the nitrogen-moiety of the amino sugars, such as N-acetylglycosamine, N-acetyl-galactosamine, etc.); ¹³C— or ²H-fatty acids, ¹³C— or ²H-ketone bodies, ¹³C-glucose, ¹³C-fructose, ¹⁸O₂, ¹³C— or ²H-acetate (incorporated into the acetyl moiety of N-acetyl-sugars, such as N-acetyl-glucosamine or N-acetyl-galactosamine), and ¹⁸O or ³⁵S-labeled sulfate (incorporated into the sulfate moiety of chondroitin-sulfate, heparan-sulfate, keratan-sulfate, and other sulfate moieties). All isotope labeled precursors may be purchased commercially, for example, from Cambridge Isotope Labs (Andover, Mass.).

(e) Precursors of Carbohydrates

Labeled precursors of carbohydrates may include any precursor of carbohydrate biosynthesis known in the art. These precursor molecules include but are not limited to H₂O, monosaccharides (including glucose, galactose, mannose, fucose, glucuronic acid, glucosamine and its derivatives, galactosamine and its derivatives, iduronic acid, fructose, ribose, deoxyribose, sialic acid, erythrose, sorbitol, adols, and polyols), fatty acids, acetate, ketone bodies, ethanol, lactate, alanine, serine, glutamine and other glucogenic amino acids, glycerol, O₂, CO₂, urea, starches, disaccharides (including sucrose, lactose, and others), glucose polymers and other polymers of the monosaccharides (including complex polysaccharides).

The precursor molecule may include labeled water, preferably ²H₂O, which is a precursor to the monosaccharides, ¹³C-labeled glucogenic precursors (including glycerol, CO₂, glucogenic amino acids, lactate, ethanol, acetate, ketone bodies and fatty acids), ¹³C— or ²H-labeled the monosaccharides, ¹³C— or ²H-labeled starches or disaccharides; other components of carbohydrates labeled with ²H or ¹³C; and ¹⁸O₂, which is a precursor to monosaccharides and complex polysaccharides.

2. Methods of Administering Labeled Precursor Molecules

Labeled precursors can be administered to a living system by various in vivo methods including, but not limited to, orally, parenterally, subcutaneously, intravenously, and intraperitoneally.

The living system may be an animal. The living system also may be human.

By way of example, in one embodiment, the labeled precursor is ²H₂0 that can be ingested (e.g., by drinking or intravenous infusion) by a living system. In another embodiment, the labeled precursor is ¹³C₁-lysine that can be ingested (e.g., by drinking or intravenous infusion) by a living system. In another embodiment, the labeled precursor is ¹³C₁-glycine that can be ingested (e.g., by drinking or intravenous infusion) by a living system. In another embodiment, the labeled precursor is ²H₃-leucine that can be ingested (e.g., by drinking or intravenous infusion) by a living system. In another embodiment, the labeled precursor is ²H₂-glucose that can be ingested (e.g., by drinking or intravenous infusion) by a living system.

The length of time for which the labeled precursor is administered may be sufficient to allow the precursor molecule to become incorporated into a biosynthetic pathway. The isotope-labeled precursor molecule also may be introduced to a living system for a period of time sufficient for the label of the isotope-labeled precursor molecule to become incorporated into one or more mitochondrial molecules and then released in the form of one or more labeled and unlabeled metabolic derivatives of the one or more mitochondrial molecules. The period of time may be a pre-determined length of time. This required duration of time may range from minutes or hours (e.g., for fast turnover mitochondrial molecules), to weeks or even months (e.g., for slow-turnover mitochondrial molecules).

The precursor molecule may be continuously or repeatedly administered. Administration of the precursor can be achieved in various ways. The precursor molecule may be administered continuously or repeatedly, so that a sufficient amount of precursor is administered such that an isotopic plateau value of maximal or isotopic enrichment is approached (i.e., wherein the concentration of labeled precursor is relatively constant over time). If the continuous labeling period can be maintained for as long as 4-5 half-lives of a mitochondrial molecule, the asymptote reached and the shape of the isotope enrichment or content curve approaching this asymptote will reveal the “true precursor” isotopic enrichment or content as well as the fractional replacement rate of the mitochondrial molecule product. By labeling to plateau while maintaining a stable precursor pool enrichment, it is thereby possible to overcome the biological complexities of cellular metabolite pools.

The precursor molecule may be administered discontinuously. For the discontinuous labeling method, an amount of labeled precursor molecule is measured and then administered, one or more times, and then the exposure to labeled precursor molecule is discontinued and wash-out of labeled precursor molecule from body precursor pool is allowed to occur. The time course of mitochondrial molecule breakdown may then be monitored by measurement of the loss of label or decay of label incorporation (dilution or die-away) in the metabolic derivative of the biological sample.

After administration of isotopically labeled water or other isotopically labeled precursor subunit molecules to a subject, the isotope is generally incorporated into a mitochondrial molecule. Examples of mitochondrial molecules include, but are not limited to, DNA, RNA, proteins, and lipids (e.g., phospholipids).

The methods of this invention are typically carried out in mammalian subjects, preferably humans. Mammals include, but are not limited to, primates, farm animals, sport animals, mice, and rats. If desired, however, the isotopically labeled precursor subunit molecule (including labeled water) may be used in an in vitro system, e.g., to contact a culture of cells or tissue. In this variation, the method for assessing metabolic fitness of the cultured cells or tissue includes: 1) contacting the cell or tissue with labeled water or other isotopically labeled precursor subunit; 2) allowing sufficient time for the label to be incorporated into a newly synthesized mitochondrial molecule; 3) isolating the mitochondria and/or a mitochondrial molecule from the cultured cell or tissue; 4) measuring isotopic content and/or pattern or rate of change of isotopic content and/or pattern of the mitochondrial molecule; and 5) calculating the rate of synthesis or rate of degradation of the mitochondrial molecule.

The labeled water or other isotopically labeled precursor subunits are generally administered at a predetermined volume and isotope concentration. Isotope concentration typically varies depending on the purpose, e.g., initiating the administration protocol of the isotopically labeled precursor subunit (i.e., “priming” the subject) or maintenance of the administration protocol of the isotopically labeled precursor subunit (i.e., “constant administration” to the subject). When given as a primer, deuterated water, for example, may be administered to achieve a sufficient concentration range in body water. Additionally, for maintenance purposes, water, including deuterated water, may be administered as a daily dose (e.g., 70 mL per day) or as a proportion of drinking water (e.g., 4% ²H₂O in drinking water). The labeled water or other isotopically labeled precursor subunit is optionally administered for a duration of time sufficient to achieve relatively stable or constant levels over the time period of incorporation (i.e., steady-state levels) in the cells, tissue, or organism of interest.

The administration of labeled water or other isotopically labeled precursor subunit to subjects may be orally or by parenteral routes, e.g., intravascular infusion or subcutaneous, intramuscular, or intraperitoneal injection.

B. Obtaining one or More Mitochondrial Molecules

After labeled water or an isotopically labeled precursor subunit has been administered, mitochondria are isolated from one or more cell types or one or more tissue samples of interest, by techniques well known in the art (see Collins M L, Eng S, Hoh R, Hellerstein M K. J Appl Physiol. 2003 June; 94(6):2203-11, herein incorporated by reference). Preferably, mitochondria are isolated from blood cells, e.g., platelets or white blood cells such as granulocytes and lymphocytes, or tissue such as skeletal or cardiac muscle. When isolated from blood cells, the cells may be obtained by methods such as venipuncture or needle aspiration, but is not so limited. In addition, tissue samples may be obtained by techniques including, but not limited to, needle aspiration, needle biopsy, endoscopic biopsy, open biopsy, and other surgical biopsy procedures known in art.

If necessary, the mitochondrial molecule (e.g., DNA) is converted to a form in which isotopic content and/or pattern can be measured. The isotopic content and/or pattern of the mitochondrial molecule is then determined by methods including, but not limited to, mass spectrometry, nuclear magnetic resonance spectroscopy, near infra-red laser spectroscopy, liquid scintillation counting or other methods known in the field. Optimally, the isotopic content and/or pattern in the mitochondrial molecule are compared to a reference value representing the isotopic content and/or pattern in the biosynthetic precursor pool, from which the mitochondrial molecule was synthesized in the cell, tissue, or organism. The rate of synthesis of the mitochondrial molecule may then be calculated, as described by Hellerstein et al. (1999), supra, which is herein incorporated by reference in its entirety, based on isotopic content and/or pattern and duration of exposure to the isotopically labeled precursor subunit, after correction for the isotopic content and/or pattern in the biosynthetic precursor pool, according to the precursor-product relationship; or, the rate of degradation of the mitochondrial component may be calculated, based on the time course of die-away of the isotopic content and/or pattern in the mitochondrial molecule after removal or wash-out (i.e., “chase”) of the labeled precursor subunit. The calculated rate(s) of synthesis and/or degradation of mitochondrial molecules may then be used to represent the metabolic fitness of the cell(s) or tissue(s) analyzed.

In practicing the methods of the invention, in one aspect, targeted molecules of interest are obtained from a cell, tissue, or organism according to methods known in the art. The methods may be specific to the particular mitochondrial molecule. Molecules of interest may be isolated from a biological sample.

A plurality of molecules of interest may be acquired from the cell, tissue, or organism. The one or more biological samples may be obtained, for example, by blood draw, urine collection, biopsy, or other methods known in the art. The one or more biological sample may be one or more biological fluids. The mitochondrial molecule may also be obtained from specific organs or tissues, such as muscle, liver, adrenal tissue, prostate tissue, endometrial tissue, blood, skin, and breast tissue. Molecules of interest may be obtained from a specific group of cells, such as tumor cells or fibroblast cells. Molecules of interest also may be obtained, and optionally partially purified or isolated, from the biological sample using standard biochemical methods known in the art.

The frequency of biological sampling can vary depending on different factors. Such factors include, but are not limited to, the nature of the molecules of interest, ease and safety of sampling, synthesis and breakdown/removal rates of the mitochondrial molecule, and the half-life of a chemical entity or drug agent.

The molecules of interest may also be purified partially, or optionally, isolated, by conventional purification methods including high pressure liquid chromatography (HPLC), fast performance liquid chromatography (FPLC), chemical extraction, thin layer chromatography, gas chromatography, gel electrophoresis, and/or other separation methods known to those skilled in the art.

In another embodiment, the molecules of interest may be hydrolyzed or otherwise degraded to form smaller molecules. Hydrolysis methods include any method known in the art, including, but not limited to, chemical hydrolysis (such as acid hydrolysis) and biochemical hydrolysis (such as peptidase degradation). Hydrolysis or degradation may be conducted either before or after purification and/or isolation of the molecules of interest. The molecules of interest also may be partially purified, or optionally, isolated, by conventional purification methods including high performance liquid chromatography (HPLC), fast performance liquid chromatography (FPLC), gas chromatography, gel electrophoresis, and/or any other methods of separating chemical and/or biochemical compounds known to those skilled in the art.

C. Biochemical Analysis

Presently available technologies (static methods) used to identify biological actions of agents measure only composition, structure, or concentrations of molecules in a cell or subcellular organelle (e.g., a mitochondrion) and do so at one point in time. The methods of the present invention, however, allow determination of the molecular flux rates of mitochondrial molecules (e.g., DNA, RNA, proteins, lipids) and their changes over time in a variety of disease states and in response to formal or informal exercise, specific training regimens, inactivity, bed-rest, life-style changes, or other behavioral factors or to exposure to an agent or combination of agents.

This allows for a more accurate assessment of a living system's fitness state (i.e., metabolic fitness) and/or aerobic capacity under a broad spectrum of physiological and pharmacological conditions as the synthesis or degradation of a mitochondrial molecule can be accomplished and a direct assessment of mitochondrial biogenesis can therefore be made. In contrast, a pure static measurement of mitochondrial molecules provides little useful information in assessing mitochondrial biogenesis and consequently is of little practical value in assessing a living system's fitness state (i.e., metabolic fitness) and/or aerobic capacity.

1. Mass Spectrometry

Isotopic enrichment in mitochondrial molecules can be determined by various methods such as mass spectrometry, including but not limited to gas chromatography-mass spectrometry (GC-MS), isotope-ratio mass spectrometry, GC-isotope ratio-combustion-MS, GC-isotope ratio-pyrrolysis-MS, liquid chromatography-MS, electrospray ionization-MS, matrix assisted laser desorption-time of flight-MS, Fourier-transform-ion-cyclotron-resonance-MS, and cycloidal-MS.

Mass spectrometers convert molecules such as proteins, lipids, carbohydrates, nucleic acids, and organic metabolites into rapidly moving gaseous ions and separate them on the basis of their mass-to-charge ratios. The distributions of isotopes or isotopologues of ions, or ion fragments, may thus be used to measure the isotopic enrichment in a plurality of mitochondrial molecules.

Generally, mass spectrometers include an ionization means and a mass analyzer. A number of different types of mass analyzers are known in the art. These include, but are not limited to, magnetic sector analyzers, electrospray ionization, quadrupoles, ion traps, time of flight mass analyzers, and Fourier transform analyzers.

Mass spectrometers may also include a number of different ionization methods. These include, but are not limited to, gas phase ionization sources such as electron impact, chemical ionization, and field ionization, as well as desorption sources, such as field desorption, fast atom bombardment, matrix assisted laser desorption/ionization, and surface enhanced laser desorption/ionization.

In addition, two or more mass analyzers may be coupled (MS/MS) first to separate precursor ions, then to separate and measure gas phase fragment ions. These instruments generate an initial series of ionic fragments of a protein, and then generate secondary fragments of the initial ions. The resulting overlapping sequences allows complete sequencing of the protein, by piecing together overlaying “pieces of the puzzle”, based on a single mass spectrometric analysis within a few minutes (plus computer analysis time).

The MS/MS peptide fragmentation patterns and peptide exact molecular mass determinations generated by protein mass spectrometry provide unique information regarding the amino acid sequence of proteins and find use in the present invention. An unknown protein can be sequenced and identified in minutes, by a single mass spectrometric analytic run. The library of peptide sequences and protein fragmentation patterns that is now available provides the opportunity to identify components of complex mixtures with near certainty.

Different ionization methods are also known in the art. One key advance has been the development of techniques for ionization of large, non-volatile macromolecules including proteins and polynucleotides. Techniques of this type have included electrospray ionization (ESI) and matrix assisted laser desorption (MALDI). These have allowed MS to be applied in combination with powerful sample separation introduction techniques, such as liquid chromatography and capillary zone electrophoresis.

In addition, mass spectrometers may be coupled to separation means such as gas chromatography (GC) and high performance liquid chromatography (HPLC). In gas-chromatography mass-spectrometry (GC/MS), capillary columns from a gas chromatograph are coupled directly to the mass spectrometer, optionally using a jet separator. In such an application, the gas chromatography (GC) column separates sample components from the sample gas mixture and the separated components are ionized and chemically analyzed in the mass spectrometer.

When GC/MS (or other mass spectrometric modalities that analyze ions of proteins, nucleic acids, lipids, and organic metabolites, rather than small inorganic gases) is used to measure mass isotopomer abundances of organic molecules, hydrogen-labeled isotope incorporation from isotope-labeled water is amplified 3 to 7-fold, depending on the number of hydrogen atoms incorporated into the organic molecule from isotope-labeled water in vivo.

In general, in order to determine a baseline mass isotopomer frequency distribution for the mitochondrial molecule, such a sample is taken before infusion of an isotopically labeled precursor. Such a measurement is one means of establishing in the cell, tissue or organism, the naturally occurring frequency of mass isotopomers of the mitochondrial molecule. When a cell, tissue or organism is part of a population of subjects having similar environmental histories, a population isotopomer frequency distribution may be used for such a background measurement. Additionally, such a baseline isotopomer frequency distribution may be estimated, using known average natural abundances of isotopes. For example, in nature, the natural abundance of ¹³C present in organic carbon is 1.11%. Methods of determining such isotopomer frequency distributions are discussed below. Typically, samples of the mitochondrial molecule are taken prior to and following administration of an isotopically labeled precursor to the subject and analyzed for isotopomer frequency as described below.

2. Measuring Relative and Absolute Mass Isotopomer Abundances

Measured mass spectral peak heights, or alternatively, the areas under the peaks, may be expressed as ratios toward the parent (zero mass isotope) isotopomer. It is appreciated that any calculation means which provide relative and absolute values for the abundances of isotopomers in a sample may be used in describing such data, for the purposes of the present invention.

3. Calculating Labeled. Unlabeled Proportion of Molecules of Interest

The proportion of labeled and unlabeled molecules of interest is then calculated. The practitioner first determines measured excess molar ratios for isolated isotopomer species of a molecule. The practitioner then compares measured internal pattern of excess ratios to the theoretical patterns. Such theoretical patterns can be calculated using the binomial or multinomial distribution relationships as described in U.S. Pat. Nos. 5,338,686, 5,910,403, and 6,010,846, which are hereby incorporated by reference in their entirety. The calculations may include Mass Isotopomer Distribution Analysis (MIDA). Variations of Mass Isotopomer Distribution Analysis (MIDA) combinatorial algorithm are discussed in a number of different sources known to one skilled in the art. The method is further discussed by Hellerstein and Neese (1999), as well as Chinkes, et al. (1996), and Kelleher and Masterson (1992), and U.S. patent application Ser. No. 10/279,399, all of which are hereby incorporated by reference in their entirety.

In addition to the above-cited references, calculation software implementing the method is publicly available from Professor Marc Hellerstein, University of California, Berkeley.

The comparison of excess molar ratios to the theoretical patterns can be carried out using a table generated for a molecule of interest, or graphically, using determined relationships. From these comparisons, a value, such as the value p, is determined, which describes the probability of mass isotopic enrichment of a subunit in a precursor subunit pool. This enrichment is then used to determine a value, such as the value A_{X}*, which describes the enrichment of newly synthesized molecules for each mass isotopomer, to reveal the isotopomer excess ratio which would be expected to be present, if all isotopomers were newly synthesized.

Fractional abundances are then calculated. Fractional abundances of individual isotopes (for elements) or mass isotopomers (for molecules) are the fraction of the total abundance represented by that particular isotope or mass isotopomer. This is distinguished from relative abundance, wherein the most abundant species is given the value 100 and all other species are normalized relative to 100 and expressed as percent relative abundance. For a mass isotopomer M_{X},

$$Fractional abundance of {M}_{x} = {A}_{x} = \frac{Abundance {M}_{x}}{{\sum }_{i = 0}^{n} Abundance {M}_{i}} ,$$

where 0 to n is the range of nominal masses relative to the lowest mass (M₀) mass isotopomer in which abundances occur.

$$\begin{matrix}\begin{matrix}Δ Fractional abundance \\ ( enrichment or depletion )\end{matrix} = {( {A}_{x} )}_{e} - {( {A}_{x} )}_{b} \\ = {( \frac{Abundance {M}_{x}}{{\sum }_{i = 0}^{n} Abundance {M}_{i}} )}_{e} - \\ {( \frac{Abundance {M}_{x}}{{\sum }_{i = 0}^{n} Abundance {M}_{i}} )}_{b} ,\end{matrix}$$

where subscript e refers to enriched and b refers to baseline or natural abundance

In order to determine the fraction of the molecules that were actually newly. synthesized during a period of precursor administration, the measured excess molar ratio (EM_{X}) is compared to the calculated enrichment value, A_{X}*, which describes the enrichment of newly synthesized biopolymers for each mass isotopomer, to reveal the isotopomer excess ratio which would be expected to be present, if all isotopomers were newly synthesized.

4. Calculating Molecular Flux Rates

The method of determining rate of synthesis includes calculating the proportion of mass isotopically labeled subunit present in the molecular precursor pool, and using this proportion to calculate an expected frequency of a molecule of interest containing at least one mass isotopically labeled subunit. This expected frequency is then compared to the actual, experimentally determined isotopomer frequency of the molecule of interest. From these values, the proportion of the molecule of interest which is synthesized from added isotopically labeled precursors during a selected incorporation period can be determined. Thus, the rate of synthesis during such a time period is also determined.

A precursor-product relationship may then be applied. For the continuous labeling method, the isotopic enrichment is compared to asymptotic (i.e., maximal possible) enrichment and kinetic parameters (e.g., synthesis rates) are calculated from precursor-product equations. The fractional synthesis rate (kₛ) may be determined by applying the continuous labeling, precursor-product formula:
kₛ=[−ln(1−f)]/t,

where f=fractional synthesis=product enrichment/asymptotic precursor/enrichment

and t=time of label administration of contacting in the system studied.

For the discontinuous labeling method, the rate of decline in isotope enrichment is calculated and the kinetic parameters of the molecules of interest are calculated from exponential decay equations. In practicing the method, biopolymers are enriched in mass isotopomers, preferably containing multiple mass isotopically labeled precursors. These higher mass isotopomers of the molecules of interest, e.g., molecules containing 3 or 4 mass isotopically labeled precursors, are formed in negligible amounts in the absence of exogenous precursor, due to the relatively low abundance of natural mass isotopically labeled precursor, but are formed in significant amounts during the period of molecular precursor incorporation. The molecules of interest taken from the cell, tissue, or organism at the sequential time points are analyzed by mass spectrometry, to determine the relative frequencies of a high mass isotopomer. Since the high mass isotopomer is synthesized almost exclusively before the first time point, its decay between the two time points provides a direct measure of the rate of decay of the molecule of interest.

Preferably, the first time point is long enough after administration of precursor has ceased, depending on mode of administration, to ensure that the proportion of mass isotopically labeled subunit has decayed substantially from its highest level following precursor administration. In one embodiment, the following time points are typically 1-4 hours after the first time point, but this timing will depend upon the replacement rate of the biopolymer pool.

The rate of decay of the molecule of interest is determined from the decay curve for the three-isotope molecule of interest. In the present case, where the decay curve is defined by several time points, the decay kinetic can be determined by fitting the curve to an exponential decay curve, and from this, determining a decay constant.

Breakdown rate constants (k_{d}) may be calculated based on an exponential or other kinetic decay curve:
k_{d}=[−ln f]/t.

As described, the method can be used to determine subunit pool composition and rates of synthesis and decay for substantially any biopolymer which is formed from two or more identical subunits which can be mass isotopically labeled. Other well-known calculation techniques and experimental labeling or de-labeling approaches can be used (e.g., see Wolfe, R. R. Radioactive and Stable Isotope Tracers in Biomedicine: Principles and Practice of Kinetic Analysis. John Wiley & Sons; (March 1992)) for calculation flux rates of molecules and flux rates through metabolic pathways of interest.

Applications of the Inventive Methods

The methods of the present invention may be used for a variety of purposes. Primarily, the methods are used to assess the metabolic fitness of a subject.

A. Assessing Risk of Medical Conditions

Assessment the metabolic fitness of a subject can be used to assess the risk of a subject for other conditions. For example, the metabolic fitness of the subject may be used to determine the risk of that subject for medical conditions such as cardiovascular disease and diabetes mellitus, or for mortality in general. Once a particular risk has been assessed, appropriate treatment can be recommended.

B. Screening Drug Agents for Effect on Metabolic Fitness

In another variation, the methods may be employed in a subject, cell culture, or tissue culture to screen drug agents, such as candidate pharmaceutical agents, in a high-throughput manner, for their effect on metabolic fitness (i.e., ability to alter metabolic fitness by increasing or decreasing metabolic fitness, or ability to prevent changes in metabolic fitness). If a cell culture system is used, the methods of the invention can be employed to screen pharmaceutical agents or candidate pharmaceutical agents in a high throughput system. Whether used in vivo or in vitro, the effect on metabolic fitness is determined by measuring and then comparing metabolic fitness before and after administration of the drug/pharmaceutical agent or candidate drug/pharmaceutical agent. The resulting difference in metabolic fitness is the effect which the candidate drug agent has on the subject, cell, or tissue of interest.

The method may be used to identify drugs that facilitate increases in metabolic fitness of a subject in response to activity. Alternatively, the method may be used to identify drugs prevent deconditioning of a subject. Use of this inventive method would help to identify a drug or candidate drug agent that prevents detraining and thereby has therapeutic utility in people forced to undergo bed-rest due to injury, illness, immobilization, or other change in metabolic fitness or aerobic demand.

The effect of a drug agent may be tested using the methods described herein. A change in the metabolic fitness or aerobic demand of a living system to which a drug agent has been administered and a living system to which a drug has not been administered identifies the drug agent as capable of altering metabolic fitness or aerobic demand of a living system. The drug agent may be administered to the same living system, or different living systems. Drug agents may be any chemical compound or composition known in the art. Drug agents include, but are not limited to, any chemical compound or composition disclosed in, for example, the 13th Edition of The Merck Index (a U.S. publication, Whitehouse Station, N.J., USA), incorporated herein by reference in its entirety.

Kits

In a further variation, the invention provides kits for performing the methods of the invention. The kits may be formed to include such components as labeled water, one or more other isotopically labeled precursor subunits, or mixtures thereof. The labeled water or other isotopically labeled precursor subunit(s) may be supplied in varying isotope concentrations and as premeasured volumes. Furthermore, the kits preferably will be packaged with instructions for use of the kit components and with instructions on how to calculate metabolic fitness.

Other kit components, such as tools for administration of labeled water or an isotopically labeled precursor subunit (e.g., measuring cup, needles, syringes, pipettes, IV tubing), may optionally be provided in the kits. Similarly, instruments for obtaining samples from the subject, cell, or tissue culture (e.g., scalpel, forceps, needles, syringes, and vacutainers) may also be optionally provided.

All publications, patents, and patent applications cited herein are hereby incorporated by reference in their entirety for all purposes to the same extent as if each individual publication, patent, or patent application were specifically and individually indicated to be so incorporated by reference. Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of the appended claims.

Applicant has not abandoned or dedicated to the public any unclaimed subject matter.

The following examples are provided to show that the methods of the invention may be used to assess metabolic fitness of cells, tissues, or organisms, including humans. Those skilled in the art will recognize that while specific embodiments have been illustrated and described, they are not intended to limit the invention.

### EXAMPLES

### Example 1

### Fractional Synthesis of Mitochondrial DNA in Rats After Isotopically Labeled Water Administration

The protocol for incorporation of ²H into rat mitochondrial DNA is illustrated in the experimental design of FIG. 1A. Male Sprague Dawley rats from Simonsen, Inc. Gilroy, Calif. were primed with 100% ²H₂O via intraperitoneal injection (a) on day zero to achieve 2% ²H₂O in body water of the rats. Deuterated water (4% ²H₂O) was then administered as drinking water to the rodents for about 10 weeks (b). There were two groups of rats: trained and untrained. The animals were then sacrificed at various timepoints (c), and tissue samples obtained from cardiac and hindlimb muscle. Thereafter, mitochondria were collected by centrifugation and mitochondrial DNA was isolated using ultracentrifugation and biochemical isolation techniques well known in the art (see Collins M L, Eng S, Hoh R, Hellerstein M K. J Appl Physiol. 2003 June; 94(6):2203-11). The DNA was hydrolyzed to free deoxyribonucleosides and derivatized using techniques known in the art (Collins et al., supra).

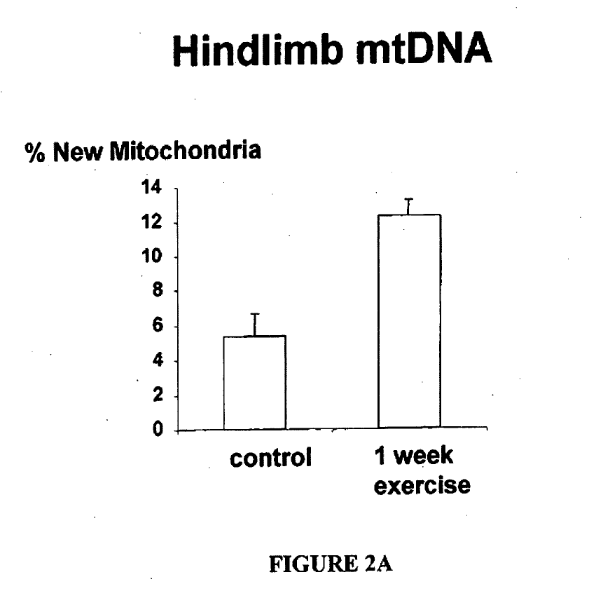

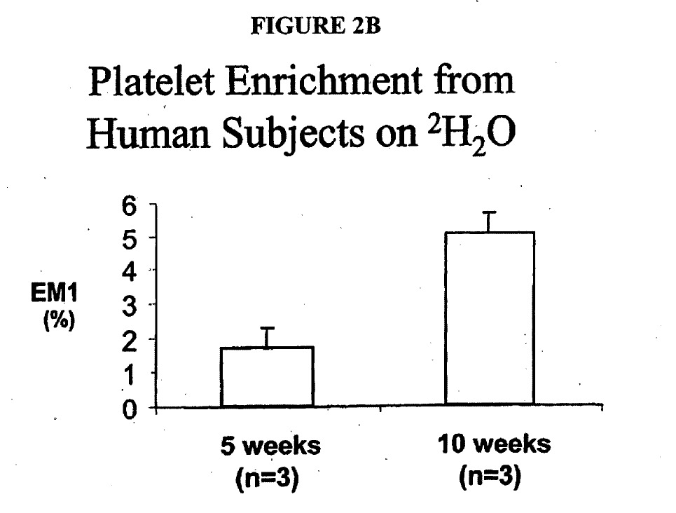

As shown in FIG. 2A, the incorporation of ²H into mitochondrial DNA was measured by gas chromatography/mass spectrometry. Animals placed on an exercise training (treadmill running) program for 1 week of exercise exhibited markedly increased incorporation of ²H into mitochondrial DNA. Conversely, sedentary, obese mice exhibited reduced mitochondrial DNA synthesis.

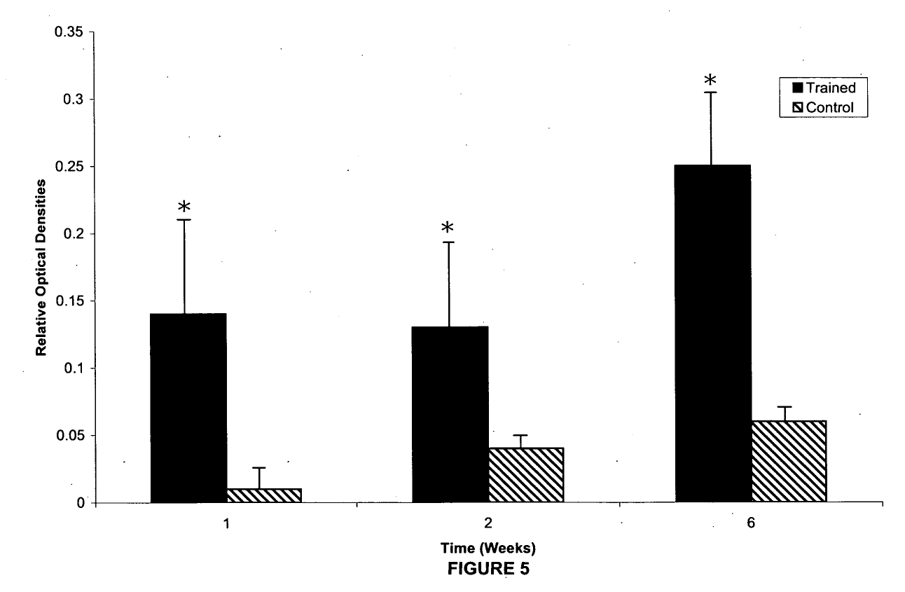

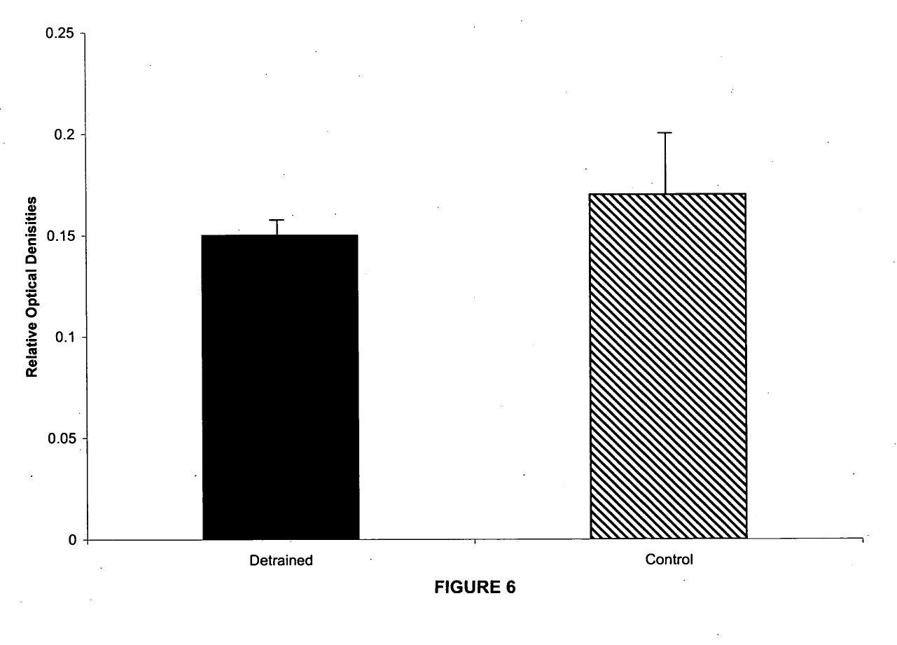

Cytochrome c oxidase subunit IV content increased with training (FIG. 5) and returned to sedentary control levels with detraining (FIG. 6). After 4 weeks of detraining, cytochrome C oxidase content in the previously trained group was not significantly different from sedentary control values, 0.15±0.01 and 0.17±0.04 mean relative optical intensities, respectively (FIG. 6). Because subunit IV is not coded by mtDNA, synthesis of new mtDNA can not directly lead to increased subunit IV content and subunit IV content does not directly represent mtDNA replication or transcription. The coordinate increases in mtDNA synthesis and subunit IV content that we observed here in both training and detraining are consistent with shared regulation by nuclear and mitochondrial elements (Williams et al., 1986). Also, if the ratio between oxidative enzymes and mtDNA content remains relatively constant in the mitochondria of a cell (Williams et al., 1986), the subunit IV content can be used as a marker of tissue mitochondrial mass, to allow fractional mtDNA synthesis to be converted to absolute biogenesis rates. Application of this technique has potential advantages over measurement of cytochrome c oxidase levels alone, since kinetic changes typically precede and are more sensitive than changes in static measures.

### Example 2

### Fractional Synthesis of Mitochondrial DNA Isolated From Human Blood Platelets After Isotopically Labeled Water Administration

The protocol for incorporation of ²H into human mitochondrial DNA from blood platelets is illustrated in the experimental design of FIG. 1B. Human subjects from the General Clinical Research Center of San Francisco General Hospital were primed with 560 ml of 70% ²H₂O by drinking 70 mls every three hours over 24 hours (a) at day zero and given 150 ml of 70% ²H₂O by drinking 50 mls 3 times a day for about 11 days. A volume of 70 ml/day of 70% ²H₂O was then administered by drinking 35 mls 2 times a day for about the next 10 weeks. Blood was drawn at various timepoints (c) and platelets isolated from the samples.

FIG. 2B shows that enrichment of platelet mitochondrial DNA from deuterated water administration increases with the increasing duration of administration of ²H₂O (Collins et al., supra).

### Example 3

### Fractional Synthesis of Mitochondrial DNA and Phospholipids Isolated From Human Muscle Biopsies After Isotopically Labeled Water Administration

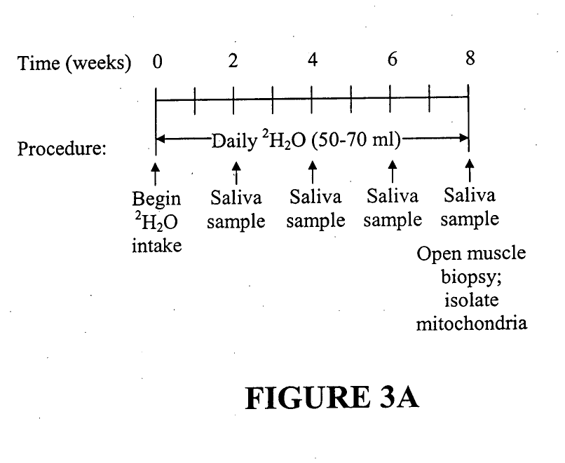

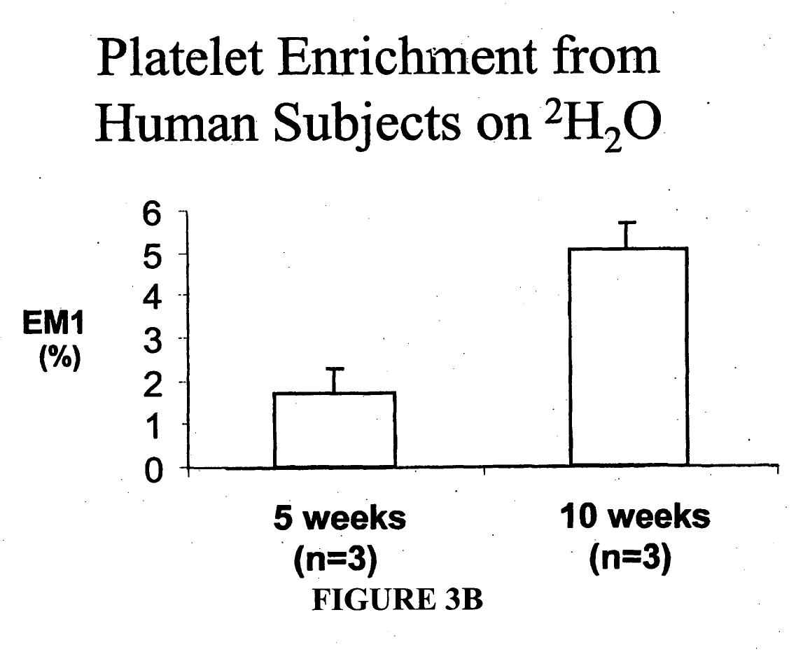

The protocol for incorporation of ²H into human mitochondrial DNA from muscle biopsy samples is illustrated in the experimental design of FIG. 3d. Five human subjects enrolled as out-patients ingested 70 ml of 70% ²H₂O three times a day for 5 days then twice a day for 5 days; then ingested 50 ml twice a day thereafter for the remainder of the eight-week study period. Every two weeks, subjects gave a saliva sample (for measurement of body ²H₂O enrichment). At week 8, an open muscle biopsy was performed under surgical conditions. Mitochondria were isolated from excised muscle tissue (1 g) by ultracentrifugation, using methods well known in the art. Isolation of mitochondrial (mt) DNA and phospholipids (PL) were by procedures well known in the art. Measurement of fractional synthesis of mt PL was as described in the general methods, supra and in Collins et al., supra. Measured ²H-incorporation (EM₁=excess abundance of M+1 mass isotopomer of the molecule) and fractional synthesis (f) of mt DNA and mt PL are shown in Table 1.

Mitochondrial Phospholipid

| | Mitochondrial | | | | | | Body |
|---|---|---|---|---|---|---|---|
| | DNA | | Cardiolipin | | Phosphatidylcholine | | ²H₂O |
| Subject# | EM₁ | f (%) | EM₁ | f (%) | EM₁ | f (%) | (%) |
| 251498 | 0.29 | 21.1 | 1.68 | 97 | 1.83 | 100 | 0.5 |
| 251515 | 0.13 | 2.8 | 1.08 | 19 | 0.44 | 8 | 1.7 |
| 251598 | 0.10 | 3.3 | 2.12 | 58 | 2.38 | 65 | 1.1 |
| 251748 | 0.17 | 3.1 | 3.18 | 49 | 3.40 | 53 | 2.0 |
| 251771 | 0.03 | 1.1 | 1.14 | 34 | 1.46 | 44 | 1.0 |

Variability in fractional synthesis of mt DNA and mt PL is apparent among healthy subjects and may reflect differences in exercise patterns or muscle aerobic demands. Different values for mt DNA and mt PL may reflect differential turnover of different components of human mitochondria. Ratios between mt DNA and mt PL synthesis may also provide information about exercise patterns or tissue aerobic demands.

### Example 4

### Fractional Synthesis of Mitochondrial Phospholipids in Rats After Isotopically Labeled Water Administration

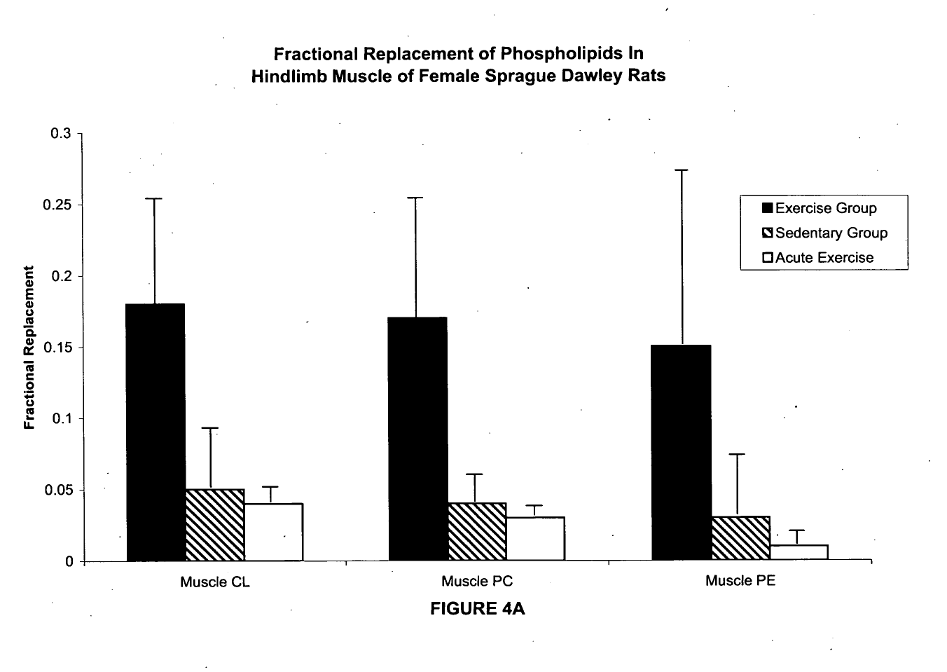

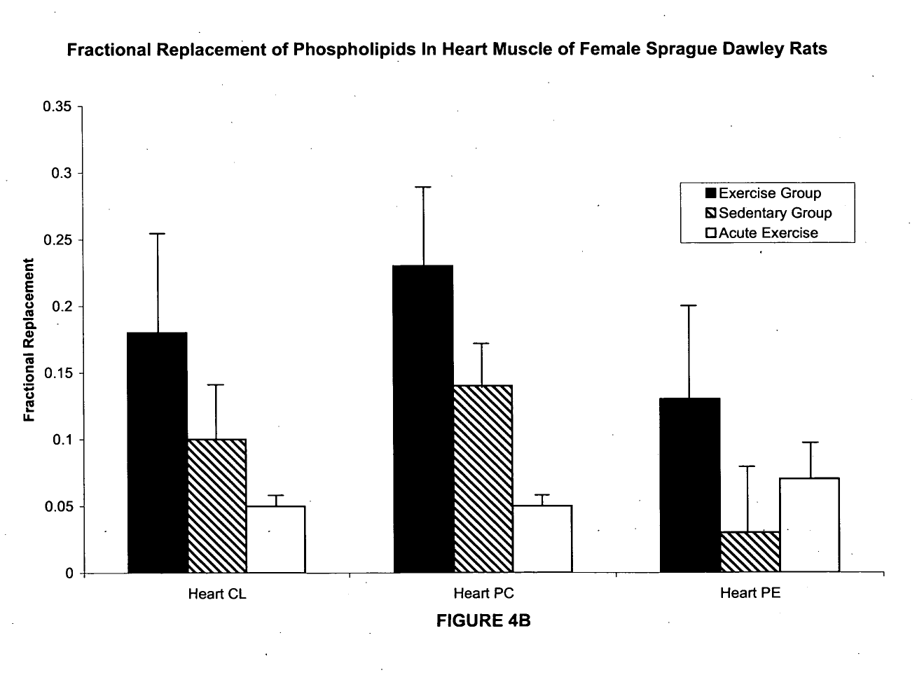

The protocol for incorporation of ²H into rat mitochondrial phospholipids is illustrated in the experimental design of FIG. 4. Female Sprague Dawley Rats from Simonsen, Inc. Gilroy, Calif. were placed into three groups, a trained group, a sedentary control, and an acute exercise group. The terms “run” and “exercise” are used synonymously in FIG. 4. The rats were primed and maintained on 4% ²H₂O as described in Example 1. After 57 days, the animals were sacrificed and tissue samples obtained from either the hindlimb muscle or cardiac muscle. Mitochondria were isolated as previously described, and assays for fractional synthesis of cardiolipin (CL), phoshphatidylcholine (PC), and phosphatidylethanolamine (PE) performed as described in Example 3, supra.

As shown in FIGS. 4A and 4B, ²H incorporation into CL, PC, and PE was the greatest in the exercise group of animals.

The results of these studies demonstrate that a laboratory test involving the drinking of deuterated water and the measurement of deuterium incorporation into molecules isolated from mitochondria, may be able to replace physiologic/whole-body exercise tests as indices of metabolic fitness and tissue oxidative needs.

### Example 5

### Persistence of Mitochondrial Biogenic Response to Voluntary or Treadmill Running

Mitochondria show plasticity in response to aerobic stimuli, but the dynamics of the mitochondrial biogenic response are not well characterized. The time course and persistence of mitochondrial biogenesis induced by voluntary and treadmill running regimens were assessed in the gastrocnemius muscle of Sprague-Dawley rats. The heavy water labeling technique for measuring mitochondrial DNA (mtDNA) synthesis was used. First, the method was tested in an exercise model known to increase mitochondrial mass (forced treadmill running). Fractional synthesis of mtDNA was significantly increased (2.3-fold, p<0.05 vs. sedentary controls) the first week of treadmill exercise and remained elevated the entire 6-week study period. Voluntary running (access to running wheels) was then compared. Voluntary exercised rats reached steady running distances of ˜10,000 m/day by the third week. Fractional synthesis of mtDNA was significantly increased the first week of exercise (3.8-fold, p<0.05), but fell to control values by the third week. No significant increase in cytochrome C oxidase activity was observed. We conclude that the normal response to voluntary running in rats involves a transient increase in mitochondrial biogenesis and that the heavy water labeling technique provides a sensitive method for exploring factors that modulate this response.

Introduction:

Mammalian systems respond to chronic increases in aerobic demand for ATP by increasing the capacity for cardiac output (Acta Physiol Scand Suppl 328: 1-45, 1968), the capillarization of skeletal muscle (Acta Physiol Scand 95: 203-205, 1975), and perhaps most important, mitochondrial mass (J Biol Chem 242: 2278-2282, 1967). Mounting evidence suggests that stimuli for the induction of mitochondrial biogenesis include lower intracellular levels of high energy phosphates (ATP and CP) and high intracellular concentrations of calcium (cf. Am J Physiol Cell Physiol 286: C1053-1061, 2004). Current understanding of mitochondrial biogenesis is that coordinated synthesis of the major components of the mitochondrial organelle occurs, including proteins, phospholipids, and mitochondrial DNA (mtDNA) (cf. Eur J Biochem 179: 275-280, 1989). The dynamics and integration of these processes are not well understood, however.

Forced treadmill training is a commonly used model to induce adaptations to increased aerobic demand. Treadmill training involves running continuously on a treadmill for 30 to 120 minutes, with increases in the duration (time) and intensity (speed) of running over a period of several weeks. This progressive overload regimen induces consistent increases in mitochondrial biogenesis as seen structurally (by electron microscopy), biochemically (by protein, phospholipid, and mtDNA content), and enzymatically (by increased catalytic activities) (cf. Am J Physiol 216: 1502-1509, 1969). The magnitude of the adaptation is dependent on the frequency, intensity, duration, and length of training (cf. J Appl Physiol 53: 844-850, 1982).

Forced exercise represents a stressor on the animals, however. Electrical or harsh auditory stimuli are often utilized to induce the animals to run. Animals also go without food and water for up to several hours at a time. Moreover, treadmill running is not a normal physiological model. The bulk of normal rodent behavior occurs during the dark cycle and consists of repeated bouts of scurrying activity (cf. Physiol Behav 43: 625-630, 1988). Forced exercise typically occurs during the light cycle and consists of prolonged periods of running.

Voluntary running is an alternative to forced treadmill exercise. Young Sprague-Dawley rats will voluntarily run between 10 to 20 km per day (cf. J Appl Physiol 79: 902-907, 1995) compared to 3.75 km run per day at the apex of treadmill training (J Biol Chem 242: 2278-2282, 1967. Voluntary-exercised animals also typically run faster than in treadmill training regimes (50-55 m/min vs. 30 m/min) (J Appl Physiol 60: 1623-1627, 1986). The primary difference between forced and voluntary training is the duration of each bout of exercise. Voluntary exercised animals run an average of 120 minutes a day, but only run between 30 seconds to 3 minutes at a time (J Appl Physiol 66: 1250-1257, 1989). The scurrying/sprinting behavior may potentially induce very different adaptations by mitochondria. Previous studies have shown variable changes in mitochondrial biogenesis in response to voluntary exercise (cf. J Appl Physiol 60: 1623-1627, 1986) and the adaptations in enzyme content or activity to voluntary exercise are more modest than with forced exercise. Because voluntary exercise more closely mimics the natural behavior of rodents and may be more relevant to public health recommendations concerning activity in humans, it is important to understand the consequences of this experimental regimen.

Here, we used a stable isotope/mass spectrometric method for measuring mtDNA synthesis (J Appl Physiol 94: 2203-2211, 2003), to characterize mitochondrial biogenesis in response to voluntary running. The time course of induced changes in mtDNA synthesis in response to this exercise regime was established in weight stable female rats and compared to the response to treadmill training.

Materials and Methods:

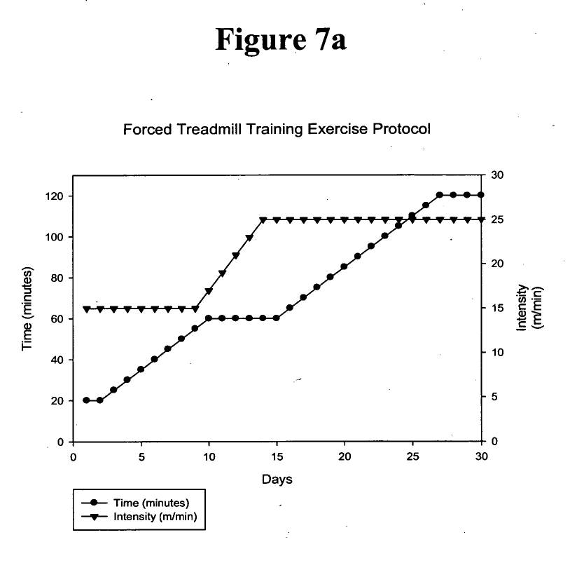

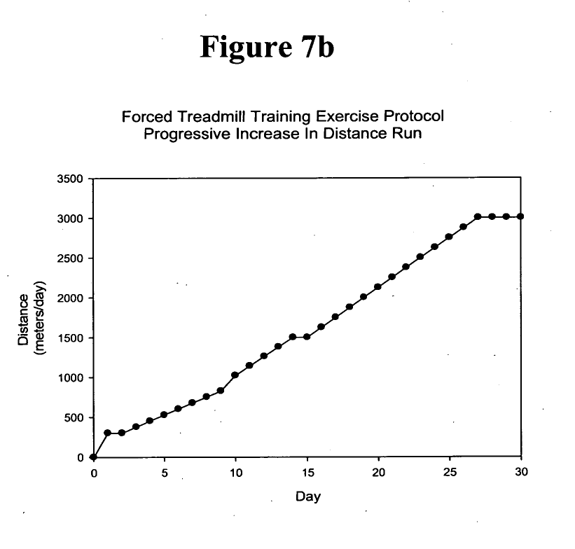

Study 1. The time-course of treadmill exercise in female rats. Two-month old female Sprague Dawley (SD) rats (Simonsen, Santa Clara, Calif.) were housed 3 animals per cage, in wire bottom cages in a climate controlled room with a 12 h: 12 h, light:dark cycle. All procedures were approved by the University of California, Berkeley Office of Laboratory Animal Care. Animals were fed Purina rodent chow ad libitum. Trained animals ran 5 days a week and were preconditioned to the treadmill (Model 42-15, Quinton Instruments, Seattle, Wash.) one week prior to the commencement of training (placed on the treadmill for 3 minutes on five consecutive days). Animals were started at a low speed (15 m/min) and low duration (20 min/day) on a treadmill with a 15% grade. Treadmill exposure (time) and intensity (speed) were gradually increased over the course of 4 weeks until the rats were running for 120 min/day at 28 m/min (FIG. 7). Incline remained constant throughout the experiment. Sedentary animals were allowed brief (five minute) exposures to the treadmill to eliminate the possibility of the treadmill itself inducing metabolic changes. Animals were labeled with heavy water during the last 2 weeks (see below).

Study #2: Voluntary exercise time-course study in female rats. Three-month-old female SD Rats (Charles River Laboratories, Wilmington, Me.) were housed individually in wire bottom cages. Animals were fed a semi-purified AIN-93M diet ad libitum (D10012M, Research Diets, New Brunswick, N.J.). Animals from the voluntary exercise group were given free access to running wheels (601943, Harvard Apparatus, Holliston, Me.) and rotations were recorded via a magnetic switch counter for 0 (n=10), 1 (n=4), 2 (n=10), 3 (n=6), or 8 weeks (n=3). Control animals did not have running wheels in their cages. Food weights were calculated by weight difference 3 times a week. Caloric intake was calculated by multiplying food weight differences by the caloric density of the food. Animals were labeled the last week of the protocol (see below).

Heavy water (²H₂O) labeling protocol. To quickly reach steady state body water enrichments, animals were injected with 99% ²H₂O containing 0.9% NaCl (Cambridge Isotopes, Andover, Mass.) (1.2 ml and 3.5 ml/100 g body weight for the treadmill training study and all other studies, respectively). Animals were subsequently provided either 8% ²H₂O drinking water ad libitum, to maintain a ˜5% body water ²H₂O enrichment, or 4% drinking water, to maintain ˜2.5% 2H₂O enrichment.

Animal sacrifice. Animals were anaesthetized with 2.5% isoflurane followed by cardiac puncture and cervical dislocation. A portion of the red gastrocnemius was quickly excised and frozen in liquid nitrogen for later enzyme activity or protein content analysis. The remaining red appearing portions of the gastrocnemius was dissected out and placed into tubes containing ice cold buffer (250 mM mannitol, 10 mM EDTA, 50 mM Tris, pH 7.4).

Mitochondria isolation by density gradient centrifugation. The red portion of the gastrocnemius was minced up into very small pieces in a Petri dish placed on ice. The small muscle pieces were homogenized with a Polytron (Brinkman, Wesbury, N.Y.) at level 5 for 45 seconds. Additional buffer was added to a level of 10 ml buffer/g tissue. The muscle homogenate was centrifuged for 10 minutes at 2000 RCF in a refrigerated centrifuge (4° C). The supernatant was filtered through sterile gauze and recentrifuged for an additional 5 minutes at 2000 RCF. The supernatant from the cleanup spin was centrifuged for 10 minutes at 8000 RCF. The supernatant was subsequently discarded and the pellet containing the mitochondria was gently broken up using a Teflon pestle. The mitochondria were again pelleted with one more 10 minute spin at 8000 RCF. The pellet was broken up in 1 ml of buffer and transferred to a microcentrifuge tube. The mitochondria were incubated for 30 minutes with 7 ul of 1 mg/ml DNase solution to get rid of any non-mitochondria DNA. The mitochondria were again pelleted by centrifugation and frozen for later mtDNA isolation.

Isolation of cells from bone marrow. The femur was perfused with a 26 gauge needle and Hanks Balanced salt solution. The solution containing the marrow was centrifuged for 3 minutes at 8000×G and the supernatant was discarded. The marrow was frozen for later analysis.

Measurement of cytochrome C oxidase activity. Frozen muscle was ground up with a motor and pestle chilled with liquid nitrogen and transferred to a 2 ml microcentrifuge tube. 1 ml of ice cold buffer (0.1M KH₂PO₄/Na₂HPO₄, 2 mM EDTA, pH 7.2) was added and the samples were homogenized for 45 seconds at speed 5 with a Powergen 700 homogenizer (Fisher Scientific). The insoluble proteins were removed via centrifugation (10 minutes, 2000 RCF, 4° C). Cytochrome C oxidase activity was assessed by the change of absorbance of ferrocytochrome C at 550 nm (CYTOCOXI, Sigma, St Louis, Mo.). Total soluble protein was measured by the Bradford assay (500-0201, BioRad, Hercules, Calif.).

Measurement of DNA enrichment. mtDNA and DNA from bone marrow were isolated with a DNeasy tissue kit (Qiagen, Valencia, Calif.) and hydrolyzed into deoxyribonucleotides. Briefly, 200 ng of DNA was incubated overnight at 37° C. in a solution containing 1.25 U acid phosphastase, 0.5 U nuclease S1, 45 mM sodium acetate, and 240 mM zinc sulfate. The deoxyribose moiety was derivitized to perfluorotetraacetate (PFTA) as described previously (22). PFTA was analyzed by negative chemical ionization using a model 5973 mass spectrometer and model 6890 gas chromatograph (Agilent, Inc., Palo Alto, Calif.). Selected ion monitoring was performed on m/z 435 and 436, representing the M₊₀ and M₊₁ ions, respectively. The excess fractional enrichment (EM₁) of dR was calculated as:

$$\begin{matrix}{EM}_{1} = \frac{{( Abundance m \text{/} z 435 )}_{sample}}{{( Abundance m \text{/} z 435 + 436 )}_{sample}} - \\ \frac{{( Abundance m \text{/} z 435 )}_{STD}}{{( Abundance m \text{/} z 435 + 436 )}_{STD}}\end{matrix}$$

where sample and STD represent the analyzed sample and unlabeled standards, respectively (21). Unlabeled PFTA standards were analyzed concurrently with samples. Calculation of fractional synthesis (f) of mtDNA was by comparison to nearly fully turned over cells (bone marrow cells), as described previously (21):

$$f ( \% ) = \frac{( EM 1 ) sample}{( EM 1 ) bone marrow} \times 100$$

Validation of mtDNA purity. Mitochondrial purity was assessed by quantitative PCR (TaqMan) as previously described (25). In brief, ˜5 ng of DNA from the mtDNA sample was reacted with 1× TaqMan Universal Mix (Applied Biosystems, Branchburg, N.J.), 200 nM β-actin forward and reverse primer, 50 nM D-loop forward and reverse primer, and 100 nM each β-actin and D-loop fluorogenic probe (50 μl reaction volume). PCR conditions were as follows: initial phase of 2 min at 50° C. followed by 10 min at 95° C. and 40 cycles of 15 seconds at 95° C. and 1 min at 60° C. Real time PCR was performed in 96-well format using the ABI Prism 7900HT and analyzed by ABI Sequence Detection System v. 2.0. The ratio of mtDNA to genomic DNA copy number was determined by the comparative CT method. Percent contamination was calculated as:

$$\begin{matrix}\% contamination = \frac{1}{+ MR \vert Normal \vert ZZMPTAG \vert} \\ MR = 6.025 \times 10 - 6 * 2 Δ CT \\ Δ CT = CT β \text{-} actin - CT D \text{-} Loop\end{matrix}$$

Statistical analysis. Data was analyzed by one-way ANOVA followed by Tukey's follow-up test (P<0.05) comparing all pair wise combinations. Two way comparisons were analyzed by T-Test. Results are represented as ±SE.

Results

Study 1: Forced (Treadmill) Exercise Time Course in Female Rats

Body Weights

Body weights were not significantly different in treadmill trained animals compared to sedentary controls (not shown).

Mitochondrial Biogenesis (Fractional Synthesis of mtDNA)

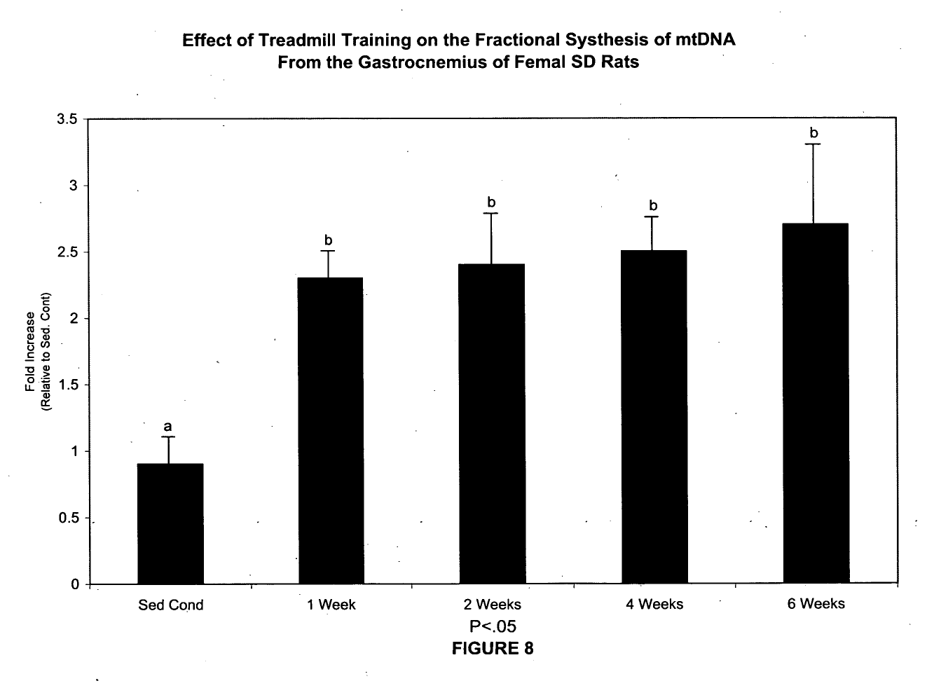

Fractional synthesis of mtDNA increased by 2.3-fold after 1 week of treadmill exercise (12.3±0.9% vs. 5.4±1.4%), even though the animals were sedentary for the first week of the 2-week labeling period. Fractional synthesis rates remained significantly elevated compared to matched sedentary control values throughout the entire 6-week training period (FIG. 8). Thus, treadmill running was confirmed to increase mitochondrial biogenesis by the heavy water labeling approach and the increase in mtDNA synthesis was persistent throughout the running program.

Study #2: Time Course of Voluntary Exercise Study in Female Rats

mtDNA Purity

Purity testing of all mtDNA samples revealed less than 10% contamination by genomic DNA (data not shown).

Body Weights, Caloric Intake, and Distance Run

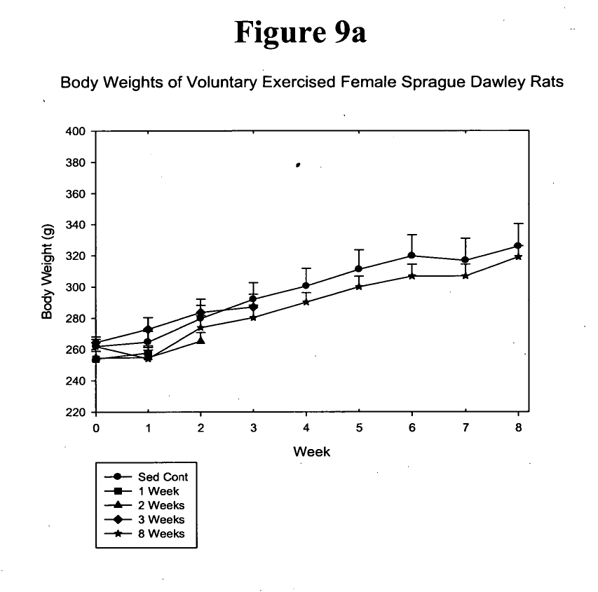

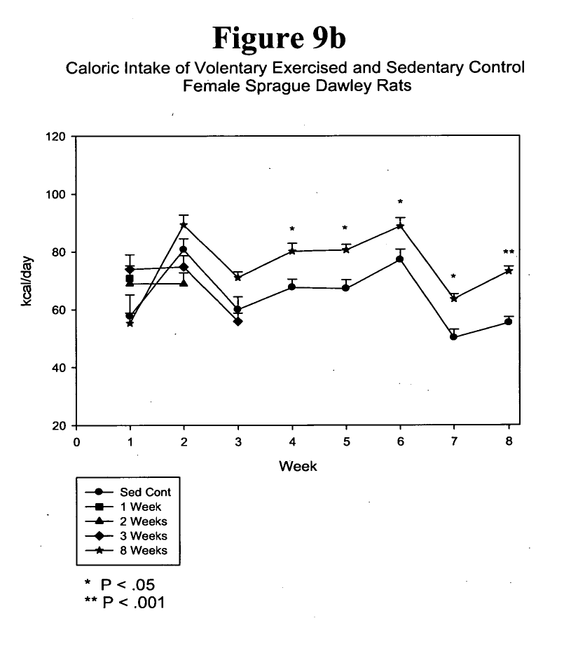

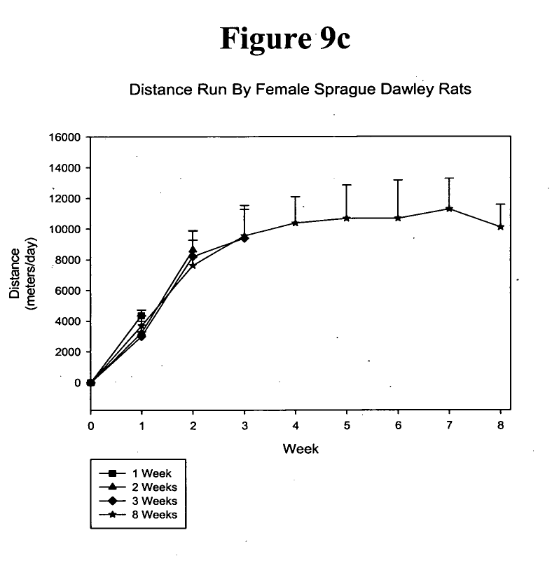

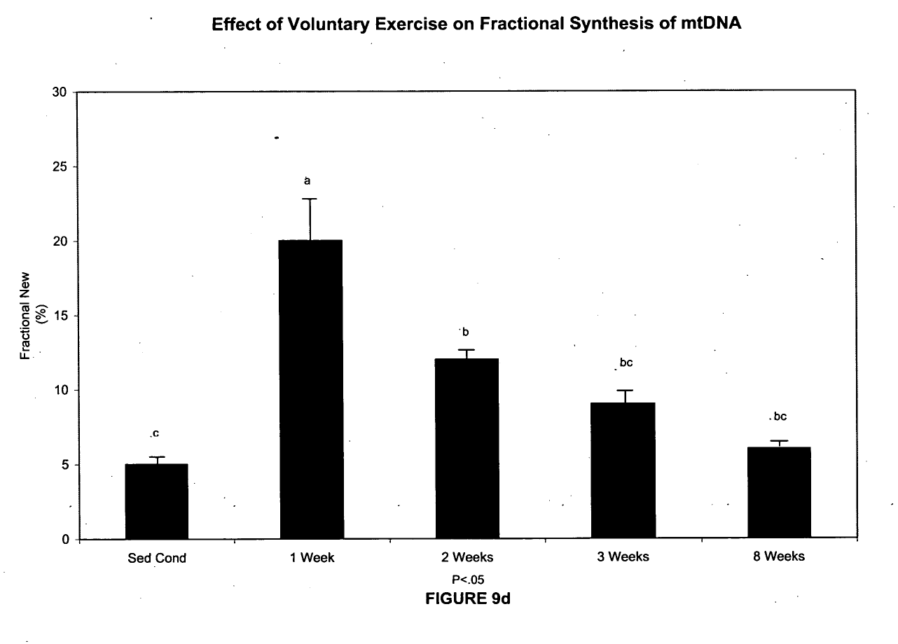

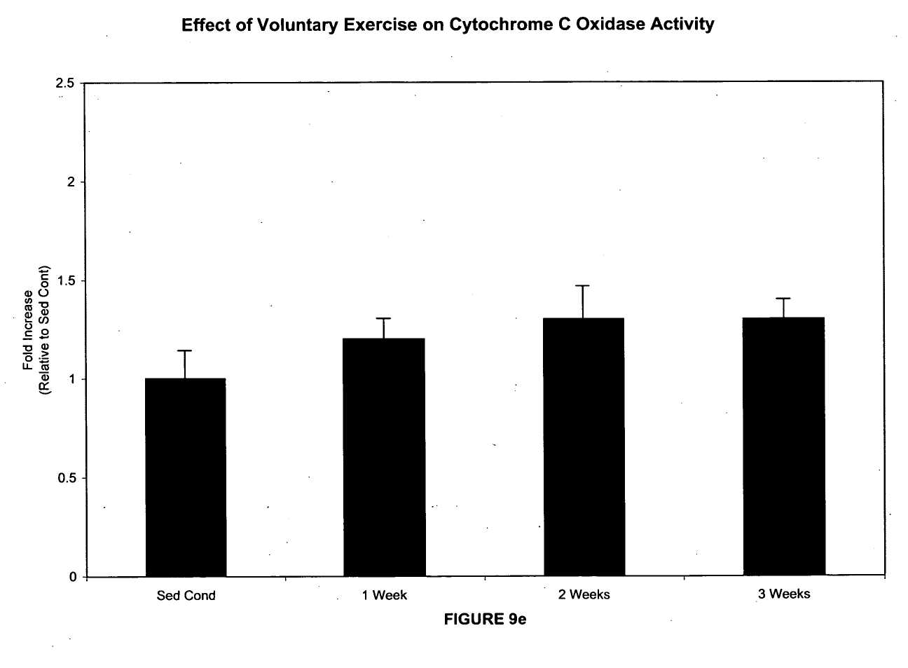

Access to running wheels did not have an effect on the body weights of female SD rats (FIG. 9A). Exercising rats maintained stable body weights relative to the sedentary control animals by eating 15 to 30% more calories after the third week of exercise (FIG. 9B). There was considerable variability within each group over time, but the 8 week exercise group consistently ate more then the sedentary group.

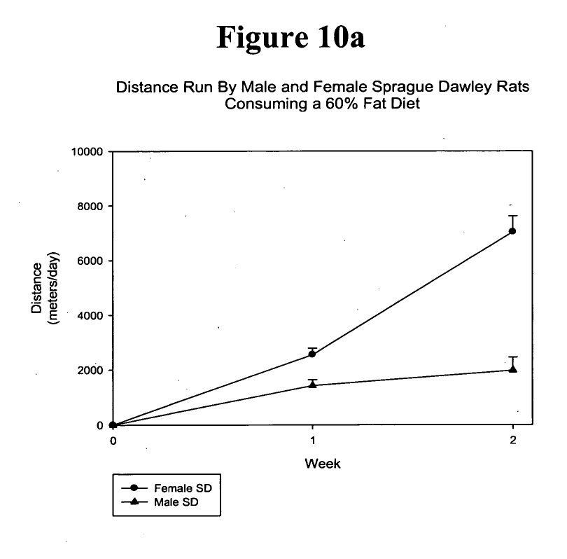

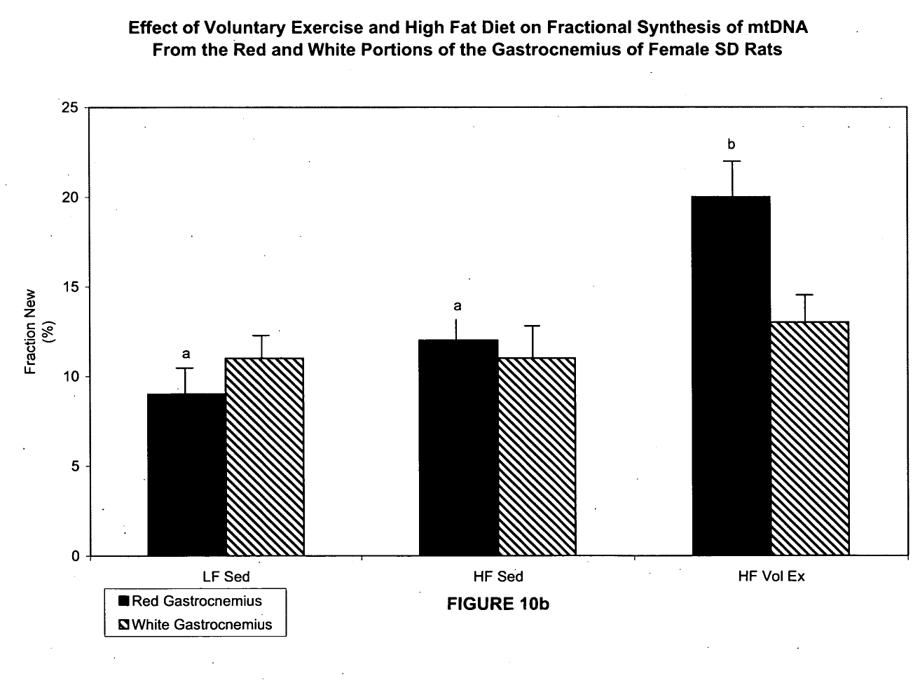

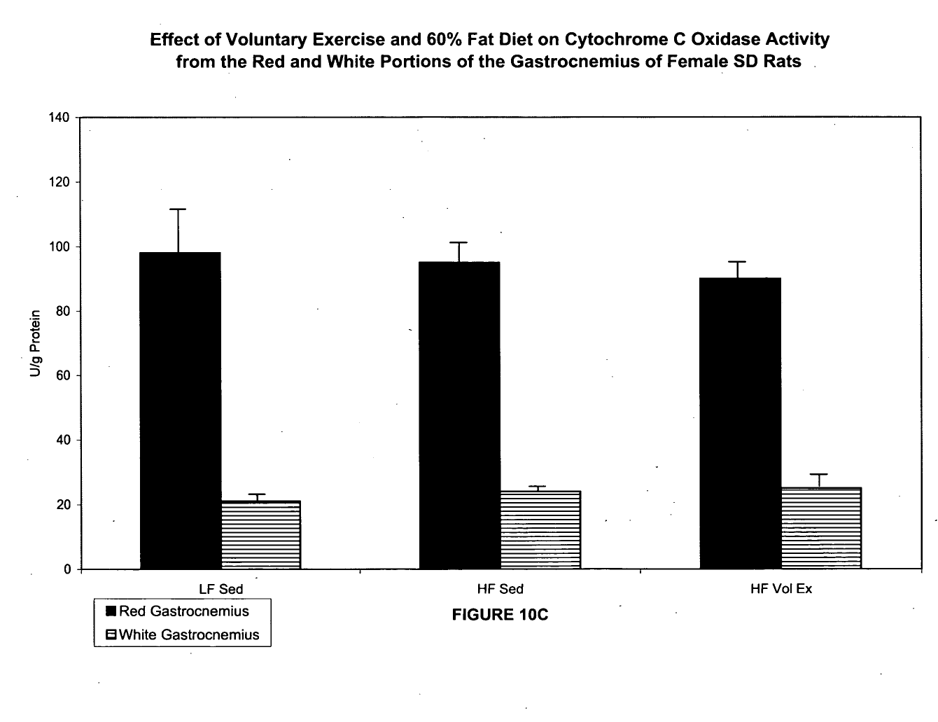

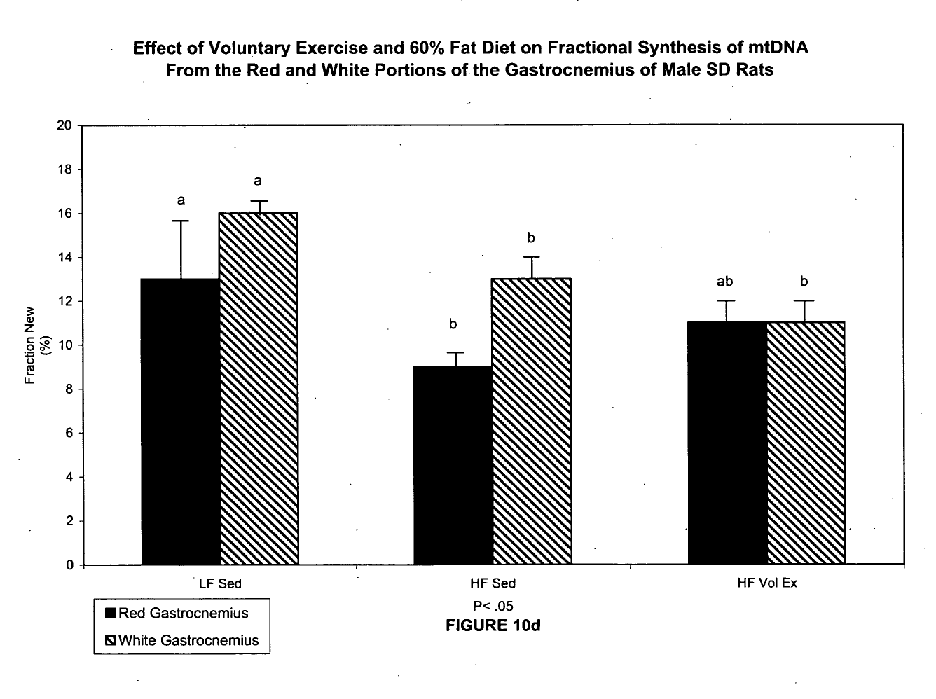

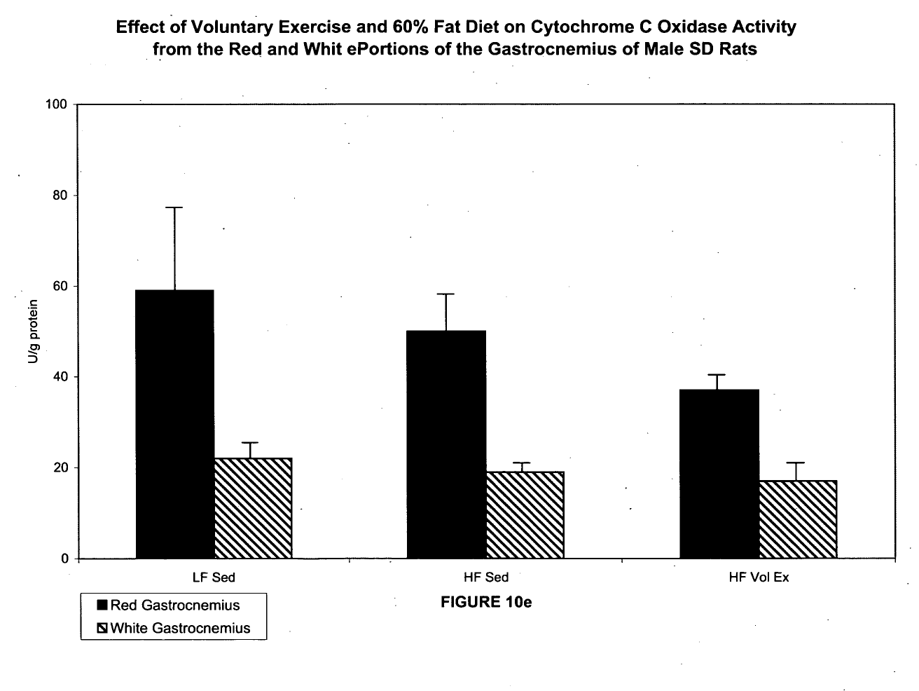

There were no significant differences in average daily running distance between any of the groups throughout the overlapping training periods (FIG. 10). Running distance reached a steady state value of ˜10,000 m/day by the third week. The greatest increase in average daily running distance was during the first two weeks of access to running wheels.

Mitochondrial Biogenesis Fractional Synthesis Rate of mtDNA) and Cytochrome C Oxidase Activity

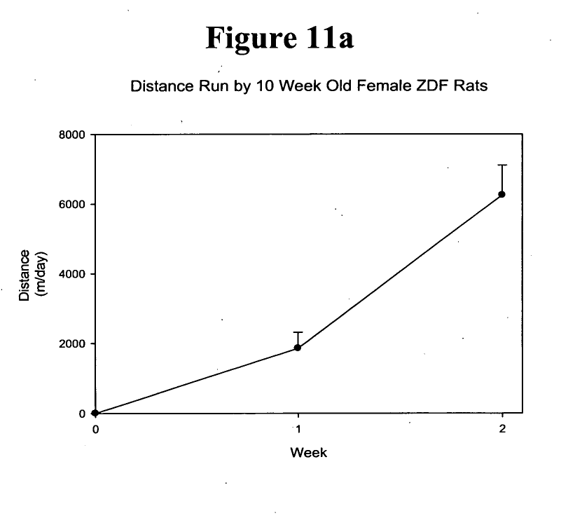

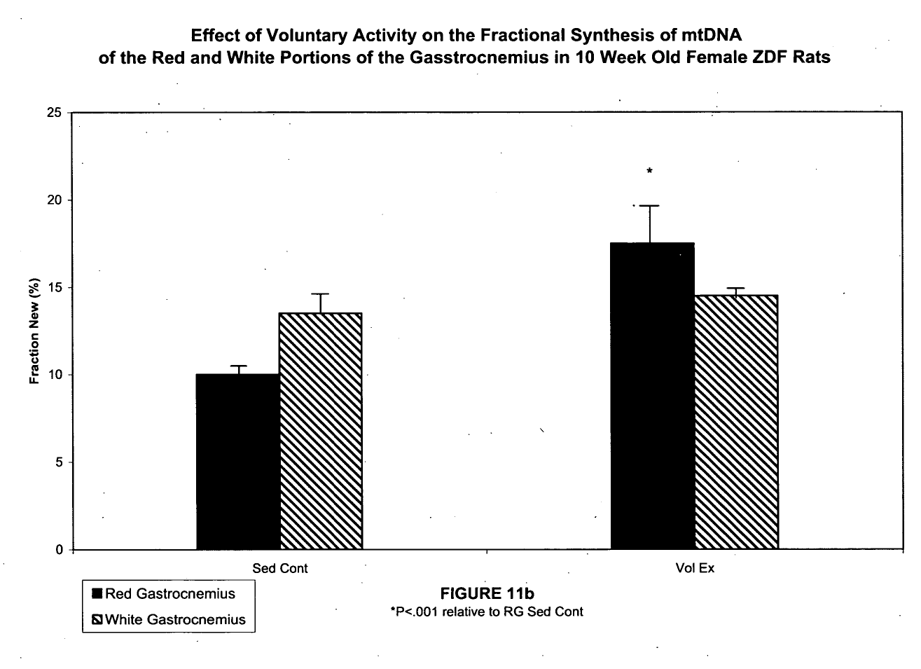

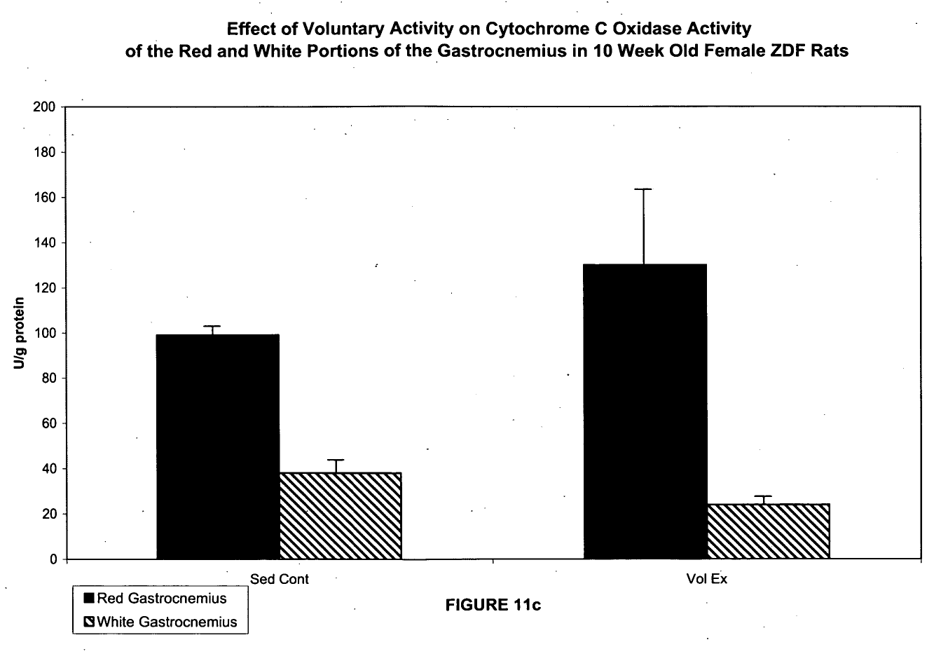

One week of voluntary wheel-running increased mtDNA fractional synthesis of mtDNA 3.8-fold relative to sedentary control animals (20.0±2.8% vs. 5.2±0.4% pre 2 weeks, respectively, FIG. 11). Fractional synthesis of mtDNA was still significantly elevated during the second week of exercise (2.3-fold elevated), but fell back to baseline values, during the third and eighth week of voluntary exercise (not significantly different from sedentary animals, FIG. 11). The calculated half life of mtDNA in sedentary animals was 87.8 days. After initiation of exercise, the estimated doubling time for mtDNA was 21.7 days.

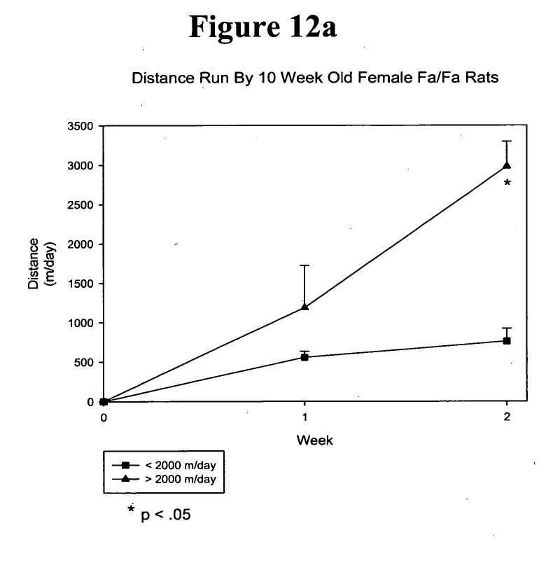

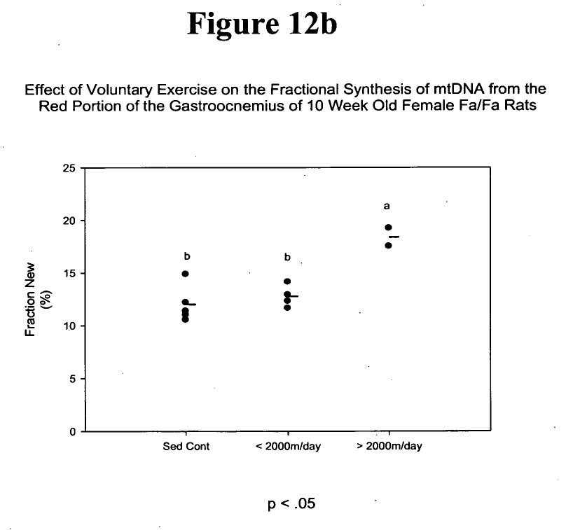

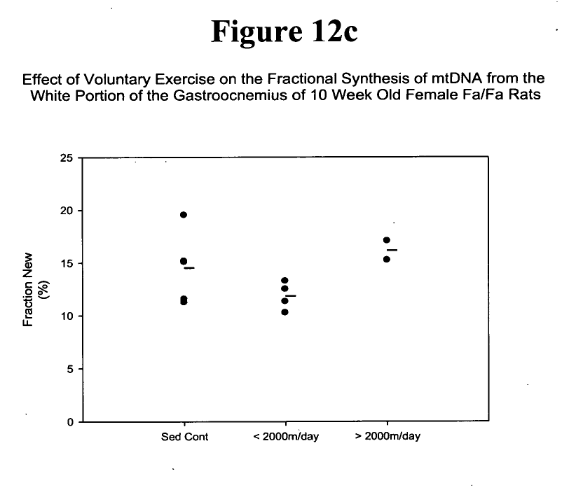

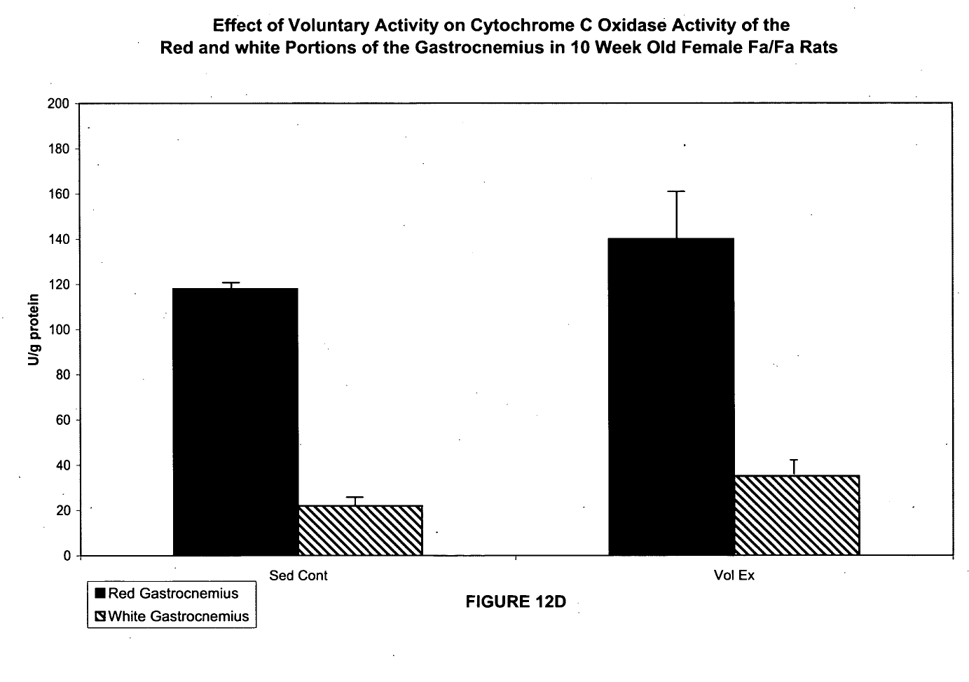

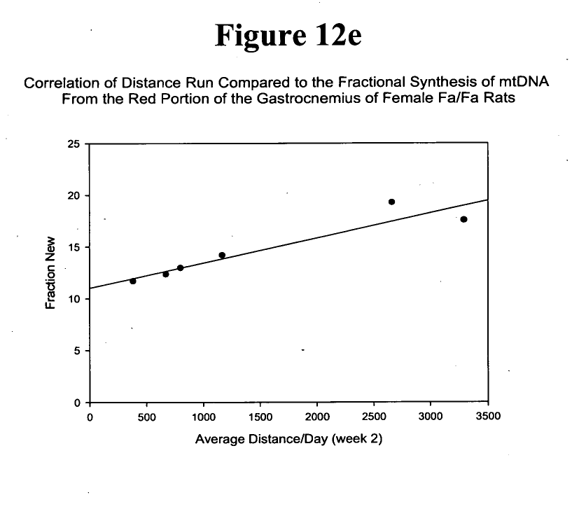

Voluntary exercise did not significantly increase cytochrome c oxidase enzyme activity relative to sedentary control animals (FIG. 12). Thus, voluntary wheel-running induced a transient increase in mtDNA synthesis during the period of increasing running distance, but this increase did not persist and did not result in increased activity of cytochrome c oxidase

Discussion:

A central element of mitochondrial biogenesis is the synthesis of new mtDNA. Measurement of deuterium incorporated from heavy water into the deoxyribose moiety of purine deoxyribonucleosides allows DNA synthesis to be quantified with excellent precision, as described previously for both genomic DNA (Biochem Biophys Res Commun 331: 203-209, 2005) and mtDNA (J Appl Physiol 94: 2203-2211, 2003). As we have discussed elsewhere (Proc Natl Acad Sci USA 99: 15345-15350, 2002), kinetic measurements are in principle capable of greater sensitivity than changes in pool sizes (static measurements). It is, for example, possible to detect a 2-fold difference in synthesis rate over a week (e.g. from 5% to 10% new mtDNA per week), whereas it would not be possible to detect the associated difference in mitochondrial pool size (105 vs. 110 units). We detected a significant increase in fractional synthesis of mtDNA in the voluntary exercise study (3.8-fold increase after 1 week of exercise), but observed no significant changes in cytochrome C oxidase activity. Cytochrome C oxidase is the terminal electron acceptor of the electron transport chain found on the inner mitochondrial membrane (Biochem J 269: 503-506, 1990) and this enzyme activity is commonly used as a marker of total mitochondrial protein content and mitochondrial mass (cf. J Biol Chem 252: 416-419, 1977). Measurement of enzyme activity had limited usefulness in the present study, however, for detecting a response to voluntary exercise. This result is consistent with previous reports of either no significant change or modest changes in mitochondrial enzyme activity, in the range between 0 to 35% increases (cf. J Appl Physiol 60: 1623-1627, 1986) after voluntary wheel-running. These observations may relate to the imprecision of the measurement or to the lack of sustained increase in mitochondrial biogenesis with voluntary wheel running (see below).

Contamination by genomic DNA accounted for less than 10% of total nucleotides in the mtDNA samples. Moreover, Collins et al. (J Appl Physiol 94: 2203-2211, 2003) showed that the replication rate of genomic DNA is typically less than or equal to that of mtDNA in weight stable female rats. Any contamination by genomic DNA should therefore result in underestimation of the fractional synthesis rate of mtDNA and should not contribute to the significant increases in mtDNA fractional synthesis that we observed during the first two weeks of voluntary exercise. The use of heavy water labeling to measure mtDNA synthesis in muscle tissue represents a sensitive approach that was able to detect reproducible, though transient, increases in mitochondrial biogenesis in this exercise model.

Fractional synthesis of mtDNA was inducible by both voluntary and forced exercise in female Sprague-Dawley rats. It is perhaps not surprising that the greatest increase in mtDNA fractional synthesis occurred during the first week of training, when the animals went from sedentary status (no exercise) to running short to moderate distances. Treadmill-trained animals had an estimated synthesis rate constant 3.8-fold higher than sedentary animals during the first week of forced running. Interestingly, this was similar to the increase that we estimate to have occurred during the first week of voluntary exercise. It will be interesting to ask whether this is the maximum physiological increase in mtDNA synthesis inducible in vivo by aerobic stimuli. The physiological limitation to synthesis of mtDNA may be transcription factors or biosynthetic precursors (e.g., dNTPs) (Gene 263: 1-16, 2001).

While both voluntary and forced exercise elicited initial increases in mtDNA synthesis, the two regimes differed in the sustainability of the response. The increased mtDNA fractional synthesis rate was sustained in the treadmill-trained animals, but was transient in the voluntary running animals. Female SD rats increased their daily wheel running from 0 m/day to 8000 m/day in the span of the first two weeks (FIG. 10) but then, after 3 weeks, the animals settled in at a new steady state in which wheel running was relatively constant (FIG. 10). Synthesis of mtDNA was not significantly elevated compared to sedentary animals, once this new steady state in running distance was reached (FIG. 11). It is of interest that the weeks with the greatest increase in mitochondrial biogenesis were those in which the greatest increase in running was also observed. A question for future research then becomes, what would happen to the persistence of mtDNA synthesis during voluntary running if the animals could be induced to continue to increase the distance run, or if increased resistance were placed on the running wheel?

Forced training appears to represent a very different stimulus than voluntary wheel running. Although the distance that the animals run per day is much less with treadmill training than voluntary exercise and the running rate is also lower (cf. J Appl Physiol 60: 1623-1627, 1986), running is continuous with treadmill training. In a general sense, these results reveal non-linearity in the relationship between total running distance or rate and mitochondrial biogenesis. The time-course is also complex. The mitochondrial biogenic response was similar during the first 2 weeks of voluntary and treadmill training, and remained high through the sixth week of treadmill training (FIGS. 8 and 12), but returned to baseline values during voluntary running by the third week. These results are consistent with the previous reports of 20% to 100% increases in mitochondrial enzyme content in treadmill-trained rats (cf. J Appl Physiol 42: 267-272, 1977) but lower and variable effects of voluntary running on mitochondrial enzyme content (cf. J Appl Physiol 60: 1623-1627, 1986). The availability of a relatively simple, high-throughput tool for measuring mtDNA synthesis may allow the nature of the relationship between running and mitochondrial biogenesis to be explored in detail.

In summary, we describe here a sensitive model and method for measuring changes in mtDNA synthesis. The model requires only one week of wheel running, is relatively simple to carry out in practice, requires less investigator time than treadmill training, and exhibits robust (though transient) increases in mtDNA synthesis. Factors that influence the initial mitochondrial biogenic response to voluntary exercise or that may extend its duration can be explored by use of this stable isotope labeling approach. This model may also prove useful for developing therapeutic approaches and agents that increase the mitochondrial biogenic response to exercise.

### Example 6

### Assessing Deconditioning of a Human

The metabolic fitness of an individual with a regular exercise training regimen may be measured by administering heavy water to achieve a total body water enrichment of ²H₂O in the range of 1.0-2.0%.

Human subjects may be primed with 560 ml of 70% ²H₂O by drinking 70 mls every three hours over 24 hours (a) at day zero and given 150 ml of 70% ²H₂O by drinking 50 mls 3 times a day for about 11 days. A volume of 70 ml/day of 70% ²H₂O is may then be administered by drinking 35 mls 2 times a day. Blood may be drawn, platelets may be isolated from the samples and mitochondrial DNA may be isolated in order to assess metabolic fitness.

The individual continues drinking 70 ml per day of the heavy water to maintain a total body ²H₂O concentration of 1.0-2.0% and terminates the exercise training regimen. Blood may then be drawn, platelets may then be isolated from the samples and mitochondrial DNA is then isolated at 1, 5, 10, 20, and 30 days to assess deconditioning due to the inactivity. By this means, the effects of regimens or protocols involved in rehabilitation or recovery from illness or injury or the effects of particular living conditions on aerobic fitness, conditioning level or deconditioning status, can be assessed quantitatively. Examples of such living conditions or protocols include bed-rest after a cerebrovascular accident or hip fracture; reduced activity after a heart attack; enforced bed-rest during pregnancy; reduced activity due to pain or discomfort caused by arthritis, obesity, neuropathy or other conditions; reduced weight bearing due to weightlessness during space travel; reduced activity due to limited opportunity for exercise; or other conditions anticipated to reduce aerobic fitness. Moreover, the effects of interventions intended to prevent or reverse deconditioning can be evaluated by use of this approach.

### REFERENCES

The following references cited herein are hereby incorporated by reference in their entirety.
1. Andersen P. Capillary density in skeletal muscle of man. Acta Physiol Scand 95: 203-205, 1975.
2. Bagby G J, Johnson J L, Bennett B W, and Shepherd R E. Muscle lipoprotein lipase activity in voluntarily exercising rats. J Appl Physiol 60: 1623-1627, 1986.
3. Baldwin K M, Cooke D A, and Cheadle W G. Time course adaptations in cardiac and skeletal muscle to different running programs. J Appl Physiol 42: 267-272, 1977.
4. Booth F W and Holloszy J O. Cytochrome c turnover in rat skeletal muscles. J Biol Chem 252: 416-419, 1977.
5. Collins M L, Eng S, Hoh R, and Hellerstein M K. Measurement of mitochondrial DNA synthesis in vivo using a stable isotope-mass spectrometric technique. J Appl Physiol 94: 2203-2211, 2003.
6. Dohm G L, Beecher G R, Stephenson T P, and Womack M. Adaptations to endurance training at three intensities of exercise. J Appl Physiol 42: 753-757, 1977.
7. Dudley G A, Abraham W M, and Terjung R L. Influence of exercise intensity and duration on biochemical adaptations in skeletal muscle. J Appl Physiol 53: 844-850, 1982.
8. Eikelboom R and Mills R. A microanalysis of wheel running in male and female rats. Physiol Behav 43: 625-630, 1988.
9. Ekblom B. Effect of physical training on oxygen transport system in man. Acta Physiol Scand Suppl 328: 1-45, 1968.
10. Fitts R H, Booth F W, Winder W W, and Holloszy J O. Skeletal muscle respiratory capacity, endurance, and glycogen utilization. Am J Physiol 228: 1029-1033, 1975.
11. Freyssenet D, Irrcher I, Connor M K, Di Carlo M, and Hood D A. Calcium-regulated changes in mitochondrial phenotype in skeletal muscle cells. Am J Physiol Cell Physiol 286: C1053-1061, 2004.
12. Garesse R and Vallejo C G. Animal mitochondrial biogenesis and function: a regulatory cross-talk between two genomes. Gene 263: 1-16, 2001.
13. Gollnick P D and King D W. Effect of exercise and training on mitochondria of rat skeletal muscle. Am J Physiol 216: 1502-1509, 1969.
14. Halseth A E, Fogt D L, Fregosi R F, and Henriksen E J. Metabolic responses of rat respiratory muscles to voluntary exercise training. J Appl Physiol 79: 902-907, 1995.
15. Harms S J and Hickson R C. Skeletal muscle mitochondria and myoglobin, endurance, and intensity of training. J Appl Physiol 54: 798-802, 1983.
16. Henriksen E J and Halseth A E. Early alterations in soleus GLUT-4, glucose transport, and glycogen in voluntary running rats. J Appl Physiol 76: 1862-1867, 1994.
17. Hickson R C. Skeletal muscle cytochrome c and myoglobin, endurance, and frequency of training. J Appl Physiol 51: 746-749, 1981.
18. Holloszy J O. Biochemical adaptations in muscle. Effects of exercise on mitochondrial oxygen uptake and respiratory enzyme activity in skeletal muscle. J Biol Chem 242: 2278-2282, 1967.
19. Hood D A. Co-ordinate expression of cytochrome c oxidase subunit III and VIc mRNAs in rat tissues. Biochem J 269: 503-506, 1990.
20. Hood D A, Zak R, and Pette D. Chronic stimulation of rat skeletal muscle induces coordinate increases in mitochondrial and nuclear mRNAs of cytochrome-c-oxidase subunits. Eur J Biochem 179: 275-280, 1989.
21. Kim S J, Turner S, Killion S, and Hellerstein M K. In vivo measurement of DNA synthesis rates of colon epithelial cells in carcinogenesis. Biochem Biophys Res Commun 331: 203-209, 2005.
22. Misell L M, Holochwost D, Boban D, Santi N, Shefi S, Hellerstein M K, and Turek P J. A stable isotope-mass spectrometric method for measuring human spermatogenesis kinetics in vivo. J Urol 175: 242-246; discussion 246, 2006.
23. Murakami T, Shimomura Y, Fujitsuka N, Nakai N, Sugiyama S, Ozawa T, Sokabe M, Horai S, Tokuyama K, and Suzuki M. Enzymatic and genetic adaptation of soleus muscle mitochondria to physical training in rats. Am J Physiol 267: E388-395, 1994.
24. Neese R A, Misell L M, Turner S, Chu A, Kim J, Cesar D, Hoh R, Antelo F, Strawford A, McCune J M, Christiansen M, and Hellerstein M K. Measurement in vivo of proliferation rates of slow turnover cells by 2H2O labeling of the deoxyribose moiety of DNA. Proc Natl Acad Sci USA 99: 15345-15350, 2002.
25. Nicklas J A, Brooks E M, Hunter T C, Single R, and Branda R F. Development of a quantitative PCR (TaqMan) assay for relative mitochondrial DNA copy number and the common mitochondrial DNA deletion in the rat. Environ Mol Mutagen 44: 313-320, 2004.
26. Ojuka E O. Role of calcium and AMP kinase in the regulation of mitochondrial biogenesis and GLUT4 levels in muscle. Proc Nutr Soc 63: 275-278, 2004.
27. Ojuka E O, Jones T E, Han D H, Chen M, and Holloszy J O. Raising Ca2+ in L6 myotubes mimics effects of exercise on mitochondrial biogenesis in muscle. Faseb J 17: 675-681, 2003.
28. Ojuka E O, Jones T E, Han D H, Chen M, Wamhoff B R, Sturek M, and Holloszy J O. Intermittent increases in cytosolic Ca2+ stimulate mitochondrial biogenesis in muscle cells. Am J Physiol Endocrinol Metab 283: E1040-1045, 2002.
29. Podolin D A, Wei Y, and Pagliassotti M J. Effects of a high-fat diet and voluntary wheel running on gluconeogenesis and lipolysis in rats. J Appl Physiol 86: 1374-1380, 1999.
30. Quiles J L, Huertas J R, Manas M, Ochoa J J, Battino M, and Mataix J. Dietary fat type and regular exercise affect mitochondrial composition and function depending on specific tissue in the rat. J Bioenerg Biomembr 33: 127-134, 2001.
31. Reichmann H, Hoppeler H, Mathieu-Costello O, von Bergen F, and Pette D. Biochemical and ultrastructural changes of skeletal muscle mitochondria after chronic electrical stimulation in rabbits. Pflugers Arch 404: 1-9, 1985.
32. Rodnick K J, Henriksen E J, James D E, and Holloszy J O. Exercise training, glucose transporters, and glucose transport in rat skeletal muscles. Am J Physiol 262: C9-14, 1992.
33. Rodnick K J, Reaven G M, Haskell W L, Sims C R, and Mondon C E. Variations in running activity and enzymatic adaptations in voluntary running rats. J Appl Physiol 66: 1250-1257, 1989.
34. Roussel D, Lhenry F, Ecochard L, Sempore B, Rouanet J L, and Favier R. Differential effects of endurance training and creatine depletion on regional mitochondrial adaptations in rat skeletal muscle. Biochem J 350 Pt 2: 547-553, 2000.
35. Takekura H and Yoshioka T. Specific mitochondrial responses to running training are induced in each type of rat single muscle fibers. Jpn J Physiol 39: 497-509, 1989.
36. Tanaka T, Ohira Y, Danda M, Hatta H, and Nishi I. Improved fatigue resistance not associated with maximum oxygen consumption in creatine-depleted rats. J Appl Physiol 82: 1911-1917, 1997.
37. Terjung R L. Muscle fiber involvement during training of different intensities and durations. Am J Physiol 230: 946-950, 1976.
38. Terjung R L, Winder W W, Baldwin K M, and Holloszy J O. Effect of exercise on the turnover of cytochrome c in skeletal muscle. J Biol Chem 248: 7404-7406, 1973.
39. Vailas A C, Pedrini V A, Pedrini-Mille A, and Holloszy J O. Patellar tendon matrix changes associated with aging and voluntary exercise. J Appl Physiol 58: 1572-1576, 1985.
40. Wang H, Hiatt W R, Barstow T J, and Brass E P. Relationships between muscle mitochondrial DNA content, mitochondrial enzyme activity and oxidative capacity in man: alterations with disease. Eur J Appl Physiol Occup Physiol 80: 22-27, 1999.
41. Williams R S, Salmons S, Newsholme E A, Kaufman R E, and Mellor J. Regulation of nuclear and mitochondrial gene expression by contractile activity in skeletal muscle. J Biol Chem 261: 376-380, 1986.
42. Zoll J, Koulmann N, Bahi L, Ventura-Clapier R, and Bigard A X. Quantitative and qualitative adaptation of skeletal muscle mitochondria to increased physical activity. J Cell Physiol 194: 186-193, 2003.
43. Zong H, Ren J M, Young L H, Pypaert M, Mu J, Birnbaum M J, and Shulman G I. AMP kinase is required for mitochondrial biogenesis in skeletal muscle in response to chronic energy deprivation. Proc Natl Acad Sci USA 99: 15983-15987, 2002.

1. A method for assessing metabolic fitness or aerobic demand of a living system, comprising:
a) administering ²H₂O to the living system in a manner sufficient to achieve a ²H₂O enrichment level of 0.5% to 15% of the total water in the living system;
b) allowing sufficient time for the ²H label to be incorporated into a mitochondrial molecule in the living system;
c) measuring the isotopic content, isotopic pattern, rate of change of isotopic content, or rate of change of the isotopic pattern, of the mitochondrial molecule; and
c) calculating the rate of synthesis or degradation of the mitochondrial molecule to assess the metabolic fitness or the aerobic demand of the living system.

2. The method of claim 1, wherein the ²H₂O enrichment level achieved is 1% to 10% of the total water in the living system.

3. The method of claim 1, wherein the ²H₂O is administered orally as two or more doses over time.

4. The method of claim 1, wherein the mitochondrial molecule is a deoxyribonucleic acid (DNA) molecule.

5. The method of claim 1, wherein the mitochondrial molecule is a ribonucleic acid (RNA) molecule selected from the group consisting of ribosomal RNA, transfer RNA, and messenger RNA.

6. The method of claim 1, wherein the mitochondrial molecule is a protein selected from the group consisting of a subunit of cytochrome c oxidase, a subunit of F₀ ATPase, a subunit of F₁ ATPase, a subunit of cytochrome c reductase, and a subunit of NADH-CoQ reductase.

7. The method of claim 1, wherein the mitochondrial molecules is a phospholipid selected from the group consisting of cardiolipin, phosphatidylcholine, phosphatidylethanolamine, and a mixture thereof.

8. The method of claim 1, wherein the living system is selected from the group consisting of skeletal muscle tissue cardiac muscle tissue, and adipose tissue.

9. The method of claim 1, wherein the living system is a mammal selected from the group consisting of a rodent and a human.

10. The method of claim 1, wherein the living system is a cell selected from the group consisting of a platelet and a cultured cell in a high-throughput screening assay system.

11. The method of claim 1, wherein the step of measuring isotopic content, pattern or rate of change of isotopic content, or pattern is performed by mass spectroscopy, NMR spectroscopy, or liquid scintillation counting.

12. A method of identifying a drug agent capable of altering metabolic fitness or aerobic demand of a living system, comprising:
a) assessing the metabolic fitness or aerobic demand of the living system according to claim 1;
b) administering the drug agent to said living system; and
c) re-assessing the metabolic fitness or aerobic demand of the living system according to claim 1 after the administration of the drug; and
d) comparing the metabolic fitness or aerobic demand of the living system before and after administration of the drug, wherein a change in the metabolic fitness or aerobic demand identifies the drug agent as capable of altering the metabolic fitness or aerobic demand of the living system.

13. A method of identifying a drug agent capable of altering metabolic fitness or aerobic demand of a first living system, comprising:
a) assessing the metabolic fitness or aerobic demand, according to the method of claim 1, of a first living system to which the drug agent has been administered;
b) assessing the metabolic fitness or aerobic demand, according to the method of claim 1, of a second living system to which the drug agent has not been administered; and
c) comparing the metabolic fitness or aerobic demand of the first living system and of the second living system, wherein a change in the metabolic fitness or aerobic demand of the first and second living systems identifies the drug agent as capable of altering the metabolic fitness or aerobic demand of the first living system.

14. A method for assessing deconditioning of a living system, comprising:
a) administering an isotopically labeled precursor molecule to the living system;
b) allowing for a period of time sufficient for the label of the isotopically labeled precursor molecule to be incorporated into a mitochondrial molecule in the living system;
c) measuring the isotopic content, isotopic pattern, rate of change of isotopic content, or rate of change of isotopic pattern of the isotopically labeled precursor molecule in the living system; and
d) calculating the rate of synthesis or degradation of the isotopically labeled precursor molecule to assess the initial metabolic fitness or aerobic demand of the living system;
e) subjecting the living system to a deconditioning event, resulting in a deconditioned living system;
f) administering the isotopically labeled precursor molecule to the deconditioned living system;
g) allowing for a period of time sufficient for the label of the isotopically labeled precursor molecule to be incorporated into a mitochondrial molecule in the deconditioned living system;
h) measuring the isotopic content, isotopic pattern, rate of change of isotopic content, or rate of change of isotopic pattern of the isotopically labeled precursor molecule in the deconditioned living system; and
i) calculating the rate of synthesis or degradation of the isotopically labeled precursor molecule to assess the deconditioning of the deconditioned living system.

15. The method of claim 14, wherein the isotopically labeled precursor molecule is labeled with a stable isotope selected from the group consisting of ²H-labeled glucose, ¹³C-labeled glucose, a ²H-labeled amino acid, a ¹⁵N-labeled amino acid, a ¹³C-labeled amino acid, ²H-labeled acetate, ¹³C-labeled acetate, a ²H-labeled ribonucleoside, a ¹³C-labeled ribonucleoside, a ¹⁵N-labeled ribonucleoside, a ²H-labeled deoxyribonucleoside, a ¹³C-labeled deoxyribonucleoside, a ¹⁵N-labeled deoxyribonucleoside, a ²H-labeled fatty acid, and a ¹³C-labeled fatty acid.

16. The method of claim 14, wherein the isotopically labeled precursor molecule is ²H₂O that is administered orally as two or more doses over time.

17. The method of claim 14, wherein the label of the isotopically labeled precursor is a radioactive isotope selected from the group consisting of ³H-labeled glucose, ¹⁴C-labeled glucose, a ³H-labeled amino acids, a ¹⁴C-labeled amino acid, ³H-labeled acetate, ¹⁴C-labeled acetate, a ³H-labeled ribonucleoside, a ¹⁴C-labeled ribonucleoside, a ³H-labeled deoxyribonucleoside, a ¹⁴C-labeled deoxyribonucleoside, a ³H-labeled fatty acid, and a ¹⁴C-labeled fatty acid.

18. The method of claim 14, wherein the mitochondrial molecule is a deoxyribonucleic acid (DNA) molecule.

19. The method of claim 14, wherein the mitochondrial molecule is a ribonucleic acid (RNA) molecule selected from the group consisting of ribosomal RNA, transfer RNA, and messenger RNA.

20. The method of claim 14, wherein the mitochondrial molecule is a protein is selected from the group consisting of a subunit of cytochrome c oxidase, a subunit of F₀ ATPase, a subunit of F₁ ATPase, a subunit of cytochrome c reductase, and a subunit of NADH-CoQ reductase.

21. The method of claim 14, wherein the mitochondrial molecule is a phospholipid selected from the group consisting of cardiolipin, phosphatidylcholine, phosphatidylethanolamine, and a mixture thereof.

22. The method of claim 14, wherein the living system is selected from the group consisting of skeletal muscle tissue, cardiac muscle tissue, and adipose tissue.

23. The method of claim 14, wherein the living system is a mammal selected the groups consisting of a rodent and a human.

24. The method of claim 14, wherein the living system is a cell selected from the group consisting of a platelet and a cultured cell in a high-throughput screening assay system.

25. The method of claim 14, wherein the step of measuring isotopic content, pattern or rate of change of isotopic content, or pattern is performed by mass spectroscopy, NMR spectroscopy, or liquid scintillation counting.

26. An isolated, ²H labeled mitochondrial molecule with its ²H label derived from ²H labeled water (²H₂O), wherein said isolated, ²H labeled mitochondrial molecule is produced by:
a) administering the ²H₂O to a living system wherein said ²H₂O is labeled with a detectable amount of ²H, wherein the ²H is incorporated into the mitochondrial molecule of the living system; and
b) isolating said ²H labeled mitochondrial molecule, wherein the isolated, ²H labeled mitochondrial molecule is a molecule selected from the group consisting of DNA, RNA, a subunit of cytochrome c oxidase, a subunit of F₀ ATPase, a subunit of F₁ ATPase, a subunit of cytochrome c reductase, a subunit of NADH-CoQ reductase, cardiolipin, phosphatidylcholine, and phosphatidylethanolamine.

27. A kit for assessing the metabolic fitness or aerobic demand of a living system, the kit comprising:
a) ²H₂O, and
b) instructions for use of the kit,
wherein the kit is used to assess the metabolic fitness or aerobic demand of a living system.
